(19) United States
(12) Patent Application Publication
(10) Pub. No.: US 2015/0265658 A1
(43) Pub. Date: Sep. 24, 2015

(54) AGENT FOR ACTIVATING SIRTUIN GENE CONTAINING EGG SHELL MEMBRANE INGREDIENT AND COMPOSITION USING THE SAME

(71) Applicants: The University of Tokyo, Tokyo (JP); ALMADO Inc., Tokyo (JP)

(72) Inventors: Yoriko ATOMI, Tokyo (JP); Miho SHIMIZU, Tokyo (JP); Yukio HASEBE, Tokyo (JP)

(21) Appl. No.: 14/667,247

(22) Filed: Mar. 24, 2015

Related U.S. Application Data
(63) Continuation of application No. 14/092,424, filed on Nov. 27, 2013, now abandoned.

(30) Foreign Application Priority Data
May 28, 2013 (JP) 2013-112270

(51) Int. Cl. A61K 35/57; A61Q 5/08; A61Q 19/00; A61K 8/98

(52) U.S. Cl. CPC A61K 35/57; A61K 8/982; A61Q 5/08; A61Q 19/00; A61K 2800/74; A61K 2800/412; A61K 2800/28

(57) ABSTRACT

Providing an agent for activating a sirtuin gene and an application thereof. The agent for activating a sirtuin gene is characterized to contain an egg shell membrane ingredient, for example an egg shell membrane-containing powder or soluble egg shell membrane ingredient.

### CROSS REFERENCE TO RELATED APPLICATIONS

This application is a continuation under 35 U.S.C. §120 of U.S. application Ser. No. 14/092,424, filed Nov. 27, 2013, which is incorporated herein reference and which claimed priority to Japanese Application No. 2013-112270, filed May 28, 2013, the entire content of which is also incorporated herein by reference.

This application is based upon and claims the benefit of priority from Japanese Patent Application No. 2013-112270, filed on May 28, 2013, the disclosure of which is incorporated herein in its entirety by reference.

### BACKGROUND

1. Technical Field

The present invention relates to an agent for activating a sirtuin gene containing an egg shell membrane ingredient, for example a soluble egg shell membrane ingredient (such as an egg shell membrane hydrolyzate) or an egg shell membrane-containing powder or fine powder, and an application thereof.

2. Related Art

An egg shell membrane (hereinafter also referred to as “ESM”) is a membrane inside a shell of a bird egg such as a chicken egg. The egg shell membrane has antibacterial and antimicrobial activities for protecting a developing embryo from the infection. The egg shell membrane has a mesh-like structure composed of tough fibrous protein based on type I, type V and type X collagens, glucosamine, desmosine and hyaluronan acid, and the like. These proteins contain a cysteine generally, are relatively stable to acid, alkali and protease, and are insoluble in water. Although the chicken egg shell membrane as a by-product in the food industry has been seldom used and thus been discarded, it is known that the chicken egg shell membrane has a function of promoting the regeneration of the skin and an action to promote the production of type III collagen that is especially called embryonal collagen, so that the effectiveness of the chicken egg shell membrane for the living body begins to attract attention.

It is known that the collagen in the skin decreases with age. The inventors reported that type III collagen, decorin and matrix metalloproteinase-2 (hereinafter referred to as “MMP2”) in a human skin fibroblast cultured on an alkaline hydrolyzed egg shell membrane (hereinafter also referred to as “ASESM”) bound to an artificial polymer almost in a dermal condition (a condition where cells are thinly present) are significantly increased, as compared with those in a human skin fibroblast cultured on a collagen coat and a cell culture dish in the same condition (Ohto-Fujita et al, 2011).

Sirtuin attracts attention as an important molecule in connection with metabolism, aging and various diseases. The sirtuin includes sirtuins 1 to 7 in a human and mouse, each of which exists in a specific compartment in a cell (for example, sirtuin 1 exists in the nucleus and cytoplasm, sirtuin 2 exists in the cytoplasm only, sirtuins 3, 4 and 5 exist in the mitochondria, sirtuin 6 exists in the nucleus only and sirtuin 7 exists in the nucleolus) and mainly has a deacetylating activity against a specific target protein. Because it is characteristic for the sirtuin proteins to consume NAD+ when exerting the enzymatic activity, the deacetylation and the central pathway of the metabolism are directly connected to each other. As it has been reported that sirtuins 1 and 3 affect both promotion and suppression of cancer, the sirtuin has positive and negative effects on the progress of disease. Therefore, it is very important that the expression level of sirtuin be moderately regulated, and only in this case, the extension of healthy life expectancy of an adult may be expected (Feldman, J. et al, 2012). In addition, it has been recently reported that the sirtuin that is a key molecule in aging is also involved in the recovery from the optical damage of the skin (Benavente et al, 2012).

However, an association between the sirtuin and the health of the skin has been seldom studied. In addition, a mechanism of action of the egg shell membrane ingredient or an effective method for increasing the sirtuin action has not been sufficiently elucidated. Furthermore, a substance has not been known so far that controls the expression of a plurality of sirtuin genes in a plurality of tissues of the whole body including the skin in parallel to contribute to the psychosomatic health.

The present invention, which has been made in view of the circumstances described above, aims to provide an agent for activating a sirtuin gene that is safe, is routinely used in a simple and easy manner, and affects a cell, particularly an agent for activating a plurality of sirtuin genes in parallel, and an application thereof to a pharmaceutical and/or cosmetic composition, food (supplement), food additive and the like which have multiplexed effects as a single material to maintain and/or improve the living body in a good state.

### SUMMARY

The present inventors have found that an egg shell membrane ingredient affects various cells of the whole body, particularly cells of the skin to activate various sirtuin genes, and further found that the egg shell membrane ingredient has a humidity retention effect thereby improving the water content and elasticity (mechanical, physical or chemical characteristic) of the skin, and have completed the present invention. Note that the skin used herein includes all cells included in the epidermis (horny layer, granular layer, prickle layer and basal layer), dermis, subcutaneous fat and dermal muscle, appendage (hair follicle, sebaceous gland and sweat gland), apocrine gland, capillary vessel, artery and the like.

Accordingly, the problem mentioned above is solved by the following present invention. In other words, an agent for activating a sirtuin gene of the present invention is characterized to contain an egg shell membrane ingredient, particularly an egg shell membrane-containing powder or soluble egg shell membrane ingredient (for example, a hydrolyzate of an egg shell membrane).

In one embodiment of the agent for activating a sirtuin gene of the present invention, the egg shell membrane-containing powder used is preferably a fine powder, in which the volume average particle diameter of the egg shell membrane-containing fine powder is 6 μm or less, and/or the volume maximum particle diameter thereof is 20 μm or less.

One embodiment of the agent for activating a sirtuin gene of the present invention is a gene expression regulator for one or more genes of a sirtuin 1 gene, sirtuin 2 gene, sirtuin 3 gene, sirtuin 4 gene, sirtuin 5 gene, sirtuin 6 gene and sirtuin 7 gene.

One embodiment of the agent for activating a sirtuin gene of the present invention is preferably used in at least any one application of an application to a composition for oral use, application to a composition for external use, application to a food (a supplement), application to a food additive, application to a regenerative medicine (a stem cell, iPS cell and the like) and application to a base material.

A composition used for a body such as a pharmaceutical composition and cosmetic composition of the present invention preferably contain the agent for activating a sirtuin gene of the present invention along with an excipient. In this case, it is preferable that the composition be a composition for external use for improving a mechanical, physical or chemical characteristic of a skin such as a skin elasticity and water content, or a composition for oral use for improving a mechanical, physical or chemical characteristic such as a skin elasticity and water content. The egg shell membrane ingredient is preferably a soluble egg shell membrane ingredient in the composition for external use, and an egg shell membrane-containing powder in the composition for oral use. As one embodiment of a pharmaceutical composition of the present invention, a tablet is preferable.

Another embodiment of the composition for oral use of the present invention preferably contains 5 to 40% egg shell membrane ingredient. An embodiment of the composition for external use may contain 1 to 80% soluble egg shell membrane ingredient, and preferably contains 1 to 40% soluble egg shell membrane ingredient.

A food additive of the present invention is characterized to consist of or contain the agent for activating a sirtuin gene of the present invention. In addition, a food of the present invention is characterized in that the food additive is added therein.

According to the present invention, the agent for activating a sirtuin gene containing the egg shell membrane ingredient such as soluble egg shell membrane or egg shell membrane-containing powder, and the application thereof may be provided. According to the agent for activating a sirtuin gene of the present invention, an extremely safe approach for regulating and moderately activating a sirtuin gene expression with no risk or very low risk of side effects may be provided. It is advantageous from the point of view of economy and environmental protection that the agent for activating a sirtuin gene of the present invention may be easily produced at high yield because the agent may be produced by effective use of a chicken egg shell membrane that is generally discarded without any need for a complicated process.

In addition, the agent for activating a sirtuin gene of the present invention may be, in accordance with the intended application and purpose, prepared into a suitable composition, thereby being widely applied as a functional food or pharmaceutical product such as preventive drug and therapeutic drug. These aspects of the composition may be routinely and readily used through a paint, intake or the like, by which the mechanical, physical and chemical characteristics such as water content and elasticity of the skin may be improved easily and safely. Because it has been reported that sirtuin 3 has an effect to inhibit cardiac hypertrophy (J Clin Invest. 2009 September 1, 119 (9): 2758-2771) and an effect on brain (PLoS One. 2012, 7 (11): e48225, CNS SIRT3 Expression Is Altered by Reactive Oxygen Species and in Alzheimer's Disease), the agent for activating a sirtuin gene of the present invention is expected to exert such effects by increasing sirtuins 1 to 7, particularly sirtuin 3. In particular, because the agent for activating a sirtuin gene of the present invention may activate or control the plurality of sirtuin genes in the whole body including the skin in parallel, the following effects will be achieved: (1) health maintenance and improvement by the activation of the whole body based on the moderate promotion of the sirtuin gene expression; (2) improvement of a slight deviation from the homeostatic range that is in “vaguely bad condition”; (3) direct improvement of the disease of a target cell/tissue; (4) early recovery from disease by the combination with various treatments or prevention of side effects caused by a drug such as an anticancer agent in medical practice using a conditioning effect; (5) buffering function by which a plurality of physical deficiencies are improved in parallel; and (6) prevention of and accelerated recovery from the injury due to physical fatigue in sports and the like, for example. Furthermore, because the agent for activating a sirtuin gene of the present invention is non-irritating and may be used in various forms in combination with various ingredients, in order that a further systemic effect may be obtained, the agent may be used in combination with another nourishment (food) or beauty ingredient and may be applied as a composition specialized to achieve a specific effect.

### BRIEF DESCRIPTION OF DRAWINGS

FIGS. 1-A to 1-M are diagrams representing the expression of various genes in the back of hairless mouse. The gene expression level after 0% or 10% ASESM solution was applied to the back of hairless mouse for 10 days was measured as an amount of mRNA (a relative amount when the expression level of GAPDH was assumed 1). Each numerical value represents a mean±SD in one group of 9 mice, and the *, ** and *** indicate significant differences at P<0.05, P<0.01 and P<0.001, respectively. FIG. 1-A represents the result of the expression level of type I collagen.

FIG. 1-B represents the result of the expression level of type III collagen.

FIG. 1-C represents the result of the expression level of type IV collagen.

FIG. 1-D represents the result of the expression level of MMP2.

FIG. 1-E represents the result of the expression level of MMP3.

FIG. 1-F represents the result of the expression level of Hsp47.

FIG. 1-G represents the result of the expression level of elastin.

FIG. 1-H represents the result of the expression level of decorin.

FIG. 1-I represents the result of the expression level of Has2.

FIG. 1-J represents the result of the expression level of TGF-β1.

FIG. 1-K represents the result of the expression level of TGF-β3.

FIG. 1-L represents the result of the expression level of sirtuin 1.

FIG. 1-M represents the result of the expression level of sirtuin 3.

FIG. 1-N shows diagrams representing the expression of sirtuin genes over time in the back of hairless mouse. The gene expression level in the epidermis after the solution of 1% ASESM was applied to the back of hairless mouse for 3, 5 and 10 days was measured as an amount of mRNA (a relative amount when the expression level of each gene after the correction with that of GAPDH was assumed 1 (when the solution of 0% ASESM was applied)).

FIG. 1-O shows diagrams representing the expression of epidermis water-related genes in the back of hairless mouse. The gene expression level after the solution of 0% or 10% ASESM was applied to the back of hairless mouse for 5 days was measured as an amount of mRNA (a relative amount when the expression level of GAPDH was assumed 1).

FIG. 2-A shows diagrams representing the effect of ASESM on the water content in the skin of the arm of woman. The “SEM (+)” and “SEM (−)” are the results in which an agent for external use containing 1% (W/V) ASESM and an agent for external use not containing ASESM were used, respectively. The white bar indicates the result before the experiment, the black bar indicates the result 4 weeks after the experiment, and the *** indicates a significant difference at P<0.001.

FIG. 2-B shows diagrams representing the effect of ASESM on the elasticity of the skin of the left arm of woman. The “OU” indicates outer upper arm, “IU” indicates inner upper arm, “OF” indicates outer front arm, and “IF” indicates inner front arm. The elasticity of the skin before and after the treatment for 12 weeks with lotion and cream not containing the ASESM (0% ASESM) (a control group, n=7) and lotion and cream containing 1% ASESM (n=7) was measured. The *, ** and *** indicate significant differences at P<0.05, P<0.01 and P<0.001, respectively.

FIG. 2-C shows diagrams representing the effect of ASESM on the elasticity of the skin of the right arm of woman. The *, ** and *** indicate significant differences at P<0.05, P<0.01 and P<0.001, respectively.

FIGS. 3-A to 3-D are diagrams representing the infiltration of ASESM in Epi-model with the use of Raman spectrophotometer (n=3, mean±SD). The and ** indicate significant differences at P<0.05 and P<0.01, respectively. FIG. 3-A represents the result in which the solution of 10% (W/V) ASESM in lotion (described below) was used.

FIG. 3-B represents the result in which the solution of 30% (W/V) ASESM in lotion was used.

FIG. 3-C represents the result in which the solution of 10% (W/V) ASESM in water was used.

FIG. 3-D represents the result in which the solution of 30% (W/V) ASESM in water was used.

FIGS. 4-A and 4-B are diagrams representing the infiltration of ASESM in human skin with the use of Raman spectrophotometer (n=3, mean±SD). The and ** indicate significant differences at P<0.05 and P<0.01, respectively. FIG. 4-A represents the result 2 minutes after the application.

FIG. 4-B represents the result 10 minutes after the application.

FIG. 5 is a diagram representing the radioactive concentration in blood, in accordance with the time course, of a digested and absorbed product of the tritium labeled egg shell membrane-containing powder orally administered to the mouse.

FIG. 6 shows diagrams representing the radioactive concentration in each tissue, 2, 6 and 12 hours after the administration, of a digested and absorbed product of the tritium labeled egg shell membrane-containing powder orally administered to the mouse.

### DETAILED DESCRIPTION

### Agent for Activating Sirtuin Gene

An agent for activating a sirtuin gene of the present invention contains an egg shell membrane ingredient as an active ingredient. The egg shell membrane ingredient may be any one of an egg shell membrane itself, processed product thereof and extract thereof. In addition, an egg shell membrane-containing powder or a soluble egg shell membrane ingredient (such as hydrolyzate) may be used, for example.

Any egg shell membrane which is inside a shell (an outer egg shell membrane and/or inner egg shell membrane and/or limiting membrane) of an egg from any terrestrial oviparous animal, in particular a bird may be used as the egg shell membrane containing the egg shell membrane ingredient used in the present invention. In particular, a chicken egg shell membrane is preferably used, from the point of view of the ready availability, cost and the like.

(Soluble Egg Shell Membrane Ingredient Used in the Present Invention)

The egg shell membrane ingredient used in the present invention may be a soluble ingredient in the egg shell membrane, for example a decomposed product or extracted material from the egg shell membrane. An egg shell membrane hydrolyzate may be produced according to a publicly known method, for example a method for producing a soluble egg shell membrane in which an egg shell membrane is decomposed in an alkaline aqueous organic solvent and the resultant decomposed product in liquid is neutralized and filtered (JP H06-21047 B), a method for producing a water-soluble egg shell membrane in which an egg shell membrane is treated with a protease (JP H07-110210 B), a method in which hydrolysis in an alkaline aqueous organic solvent and subsequent treatment with an anion exchange resin are performed (JP 5179847 B1) and an alkaline hydrolysis method disclosed in U.S. Pat. No. 8,211,477 (title of the invention: Solubilized protein composition obtained from eggshell membrane), and a modified method thereof.

A soluble egg shell membrane may be produced based on, other than such an alkaline hydrolysis, a picric acid-pepsin treatment method (Takahashi K, Shirai K, Kitamura M, Hattori M. Soluble egg shell membrane protein as a regulating material for collagen matrix reconstruction. Biosci Biotechnol Biochem. 1996 August; 60(8): 1299-302), an acid-pepsin hydrolysis method (F. Yi, J. Yu, Z. X. Guo, L. X. Zhang, and Q. Li, “Natural bioactive material: a preparation of soluble eggshell membrane protein,” Macromolecular Bioscience, vol. 3, no. 5, pp. 234-237, 2003; F. Yi, Z. X. Guo, L. X. Zhang, J. Yu, and Q. Li, “Soluble eggshell membrane protein: preparation, characterization and biocompatibility,” Biomaterials, vol. 25, no. 19, pp. 4591-4599, 2004; Jun Jia, Geng Liu, Jian Yu, and Yuanyuan Duan. 2012. Preparation and characterization of soluble eggshell membrane protein/PLGA electrospun nanofibers for guided tissue regeneration membrane. J. Nanomaterials 2012, Article 25 (January 2012), 1 pages. DOI=10.1155/2012/282736 http://dx.doi.org/10.1155/2012/282736), a method in which reduction of S—S bond and trypsin treatment are performed (Kodali VK, Gannon SA, Paramasivam S, Raje S, Polenova T, Thorpe C. A novel disulfide-rich protein motif from avian eggshell membranes. PLoS One. 2011 Mar. 30; 6(3): e18187. doi: 10.1371/journal.pone.0018187), and the like.

The soluble egg shell membrane ingredient may be produced according to these methods in which, instead of the egg shell membrane, an egg shell membrane-containing powder is used as described below.

As the egg shell membrane decomposed product, a commercially available one may be used. For example, an egg shell membrane hydrolyzate, product name “EM PROTEIN-P” from Kewpie Corporation, Tokyo, Japan may be used.

(Egg Shell Membrane-Containing Powder Used in the Present Invention)

The egg shell membrane-containing powder used in the present invention is not limited in particular, as long as the powder is a powder including at least the egg shell membrane, but is preferably an egg shell membrane-containing fine powder with the volume average particle diameter being 6 μm or less. In addition, it is preferable for the egg shell membrane-containing fine powder used in the present invention that the volume maximum particle diameter be 20 μm or less. Note that, in the specification of this application, “volume average particle diameter” and “volume maximum particle diameter” of the powder or fine powder refer to values measured with the use of a laser diffraction-type particle size distribution measuring instrument (LMS-30, made by Seishin Enterprise Co., Ltd.). Herein, “volume average particle diameter” refers to a particle diameter at which the cumulative value from the smaller particle diameter side is 50% in the particle size distribution. In addition, in case where the particle diameter of the egg shell membrane-containing powder or fine powder is measured, a measurement sample is used in which the egg shell membrane-containing powder or fine powder is dispersed in water by a surfactant. Note that “powder” refers to any particulate regardless of the particle size, and “fine powder” may refer to a particulate with the maximum particle diameter and/or the average particle diameter being generally less than 100 μm but does not intend to strictly distinguish particulates.

Controlling the particle size distribution of the egg shell membrane-containing fine powder such that the volume average particle diameter of the egg shell membrane-containing fine powder is 6 μm or less or the volume maximum particle diameter thereof is 20 μm or less may further improve the digestion and absorption efficiency and the sirtuin gene activation efficiency, as compared with both efficiencies according to a conventional egg shell membrane powder (with the maximum particle diameter being 100 to 200 μm) that is obtained through a classifying treatment with 70 mesh or 150 mesh.

The reason why such an effect is obtained is not sure, but is estimated as follows. Generally, the surface area per unit volume of a particle becomes larger as the particle diameter becomes smaller. Therefore, when the particle is composed of only a soluble or readily-soluble substance in a digestive fluid, the digestion and absorption efficiency is improved as the particle diameter becomes smaller, with the result that the sirtuin gene activation efficiency is expected to be improved.

However, more finely pulverizing a conventional egg shell membrane powder with the maximum particle diameter being approximately 100 to 200 μm and the average particle diameter being the order of several ten to one hundred and several ten μm such that the maximum particle diameter or the average particle diameter is changed within these particle diameter levels seldom improves the digestion and absorption efficiency and the sirtuin gene activation efficiency. This is presumably because the egg shell membrane has a tough mesh-like structure based on a fibrous protein, and thus in the egg shell membrane particle that has been pulverized within these particle diameter levels, the tough mesh-like structure is still maintained.

On the other hand, according to the egg shell membrane-containing fine powder with the volume average particle diameter being 6 μm or less or the volume maximum particle diameter being 20 μm or less, each of the digestion and absorption efficiency and the sirtuin gene activation efficiency is considerably improved, as compared with each of the efficiencies according to the conventional egg shell membrane powder. It appears that such improvements of the digestion and absorption efficiency and the sirtuin gene activation efficiency do not simply result from the fact that the particle diameter becomes small, but result from the fact that the fibrous, tough, mesh-like structure the egg shell membrane originally has is destroyed in the whole egg shell membrane fine particle during a process of finely pulverizing the egg shell membrane so that the whole egg shell membrane fine particle is more easily dissolved in a digestive fluid.

Therefore, the powder used as the egg shell membrane ingredient in the present invention may have a volume maximum particle diameter of more than 20 μm, a volume average particle diameter of more than 6 μm, or a volume maximum particle diameter of more than 20 μm and a volume average particle diameter of more than 6 μm, however, from the point of view that the digestion and absorption efficiency and the sirtuin gene activation efficiency are further improved, it is more preferable that the powder be the egg shell membrane-containing fine powder with the volume average particle diameter being 6 μm or less and/or the volume maximum particle diameter being 20 μm or less.

In the agent for activating a sirtuin gene containing the egg shell membrane-containing powder of the embodiment, at least the pulverized or finely pulverized egg shell membrane ingredient is contained, and in addition to this, a pulverized or finely pulverized egg shell calcium ingredient may be contained. In this case, it is particularly preferable that the egg shell membrane-containing powder of the embodiment be either in a form in which only the egg shell membrane ingredient is contained (first form) or in a form in which only the egg shell membrane ingredient and the egg shell calcium are contained (second form). Because in the agent for activating a sirtuin gene containing the egg shell membrane-containing powder in the first form, only the egg shell membrane ingredient is contained, the agent may be widely used for various targets, for example a pharmaceutical composition (in particular in a solid dosage form such as a tablet), food additive and the like. Note that in both of the egg shell membrane-containing powder in the first form and the egg shell membrane-containing powder in the second form, contamination with an impurity ingredient during a producing process and the like is acceptable. In addition, another nutrient and the like in addition to the egg shell membrane ingredient and the egg shell calcium ingredient may be contained in the agent for activating a sirtuin gene containing the egg shell membrane-containing powder of the embodiment.

(Method for Producing Egg Shell Membrane-Containing Powder or Fine Powder Used for Agent for Activating Sirtuin Gene of the Present Invention)

When the egg shell membrane-containing powder used in the present invention is produced, the egg shell membrane as a raw material in which the egg shell membrane is separated or a raw material in which the egg shell membrane is attached to the egg shell may be used, and the raw materials may also be used in combination with the egg shell membrane powder. A method for pulverizing such raw materials may be any one of publicly known pulverizing methods. As the egg shell membrane-containing powder, a commercially available egg shell membrane powder may be used, and as the commercially available egg shell membrane powder, for example, a product name “EM Powder 300” (made by Kewpie Corporation) may be used. When the egg shell membrane-containing fine powder is produced, a commercially available egg shell membrane powder or a commercially available egg shell membrane powder and an egg shell calcium may be used, and further this or these may be finely pulverized such that the volume average particle diameter is 6 μm or less and/or the volume maximum particle diameter is 20 μm or less.

The egg shell membrane-containing fine powder used in the present invention may be produced at least through a finely pulverizing step in which egg shell membrane-containing raw materials collide with each other in a gas to be finely pulverized. In such a finely pulverizing step, a so-called jet mill is used. Such a pulverizing method, as compared with a conventional pulverizing method in which a hard crashing member such as a rotary cutter collides with and pulverizes the raw material, seldom generates a frictional heat caused by the contact and collision between the crashing member and the raw material at the time of pulverization, thereby lessening a damage to ingredients included in the egg shell membrane, such as an amino acid and protein, which are easily denatured, degraded and decomposed by the heat. In other words, during a producing process, an active ingredient in the egg shell membrane is not easily lost. In addition to this, it is advantageous that because a high pressure gas rather than a crashing member is used to pulverize the raw material, the egg shell membrane-containing fine powder is not contaminated with an impurity derived from the pulverizer.

In the finely pulverizing step, the egg shell membrane-containing raw material is pulverized by jet mill such that the volume average particle diameter is preferably 40 μm or less, more preferably 20 μm or less, and further preferably 10 μm or less. In addition, in this case, it is preferable that the egg shell membrane-containing raw material be pulverized such that the volume maximum particle diameter is 20 μm or less. On the other hand, the lower limit of the volume average particle diameter of the egg shell membrane-containing raw material pulverized by jet mill is not limited in particular, but from the practical point of view of the productivity and the like, the volume average particle diameter is preferably 4 μm or more, and more preferably 5 μm or more.

When the egg shell membrane-containing raw material after being pulverized by jet mill has a volume maximum particle diameter of 20 μm or less and/or a volume average particle diameter of 6 μm or less, the raw material may be directly used as the agent for activating a sirtuin gene containing the egg shell membrane-containing fine powder of the present invention. On the other hand, when a coarse particle with the particle diameter being more than 20 μm in the particle size distribution is included, after the finely pulverizing step, a classifying step in which the coarse particle is classified through a sieve with an opening of 20 μm or less and then removed may be performed further.

In addition, in the method for producing the egg shell membrane-containing fine powder used in the agent for activating a sirtuin gene of the present invention, another step or process may be performed if necessary. For example, the finely pulverizing step may include a first finely pulverizing process and a second finely pulverizing process, in which a raw material powder after undergoing the first finely pulverizing process may be sterilized under high pressure steam and then treated in the second finely pulverizing process. In the process in which the egg shell membrane-containing raw material is pulverized by jet mill to be micronized, although the antibacterial property of the egg shell membrane tends to decrease, performing the sterilization as described above easily prevents the reproduction of fungi or bacteria in the egg shell membrane-containing fine powder of the embodiment.

(Composition Containing Agent for Activating Sirtuin Gene)

The composition of the present invention contains the agent for activating a sirtuin gene of the present invention and at least one type of excipient or diluent base. Because the agent for activating a sirtuin gene of the present invention is non-irritating, when the agent for activating a sirtuin gene is used as a composition such as a medicine and cosmetic product, such a composition may be any composition for oral or external use, and the dosage form thereof is not limited in particular. Blending the composition for external use such as an eye drop, nose drop, ear drop, oral medicine (collutory, aerosolized agent) and suppository (bougie, ointment, enema) with a commonly used and publicly known ingredient may prepare various dosage forms such as a liquid formulation, solid formulation and semi-solid formulation, in accordance with the intended use. Preferable examples of the composition may include lotion, ointment, gel, cream, spray, patch, powder and the like. In order that the composition may be orally administered or taken, the composition is preferably prepared into a composition for oral use in the form of a tablet, powder, granule, capsule, liquid and the like. The composition for oral use may also be in the form of a sublingual medicine (not only a tablet but also a sheet such as wafer sheet, and paste), jelly and drinkable medicine in which a fine powder is suspended. The absorption from the oral mucous membrane is advantageous, because the active ingredient directly enters the heart from the capillary vessel through the internal jugular vein, thereby avoiding the decomposition in the lumen of the gut, the metabolism, and the first pass effect by the metabolism in the liver, and then the active ingredient travels in the whole body at once. Various ingredients and producing methods for producing a composition such as a medicine and cosmetic product in various types of dosage form including the examples described above are publicly known in the field of production of medicine, cosmetic product and the like, and a person skilled in the art can appropriately select it in accordance with the situation. Note that “pharmaceutical composition” used herein is not limited for humans but includes a pharmaceutical composition for mammals such as dog and cat reared as a companion or domestic animal. In addition, “cosmetic composition” includes not only a cosmetic product but also any quasi-drug and medicinal cosmetic product under the Pharmaceutical Affairs Act, and the like.

Note that in the specification and the claims, unless otherwise indicated, “%” is a percentage in which the weight or volume of the whole composition is assumed 100%, and when the ingredient of interest is solid (powder and the like), the percentage is given by (W/V) or (W/W), and when the ingredient of interest is liquid, the percentage is given by (V/V).

The effective dose of the pharmaceutical composition for activating a sirtuin gene of the present invention varies in accordance with the type and degree of the disease or symptom to be treated or prevented, the state of the subject to be administered (including age, sex, physical state and the like), the dosage form and the like.

The oral dose of such a pharmaceutical composition to a human (an adult weighing 60 kg) in terms of the amount of the egg shell membrane ingredient is preferably 1 mg to 100,000 mg per day. Specifically, the effective dose of the oral pharmaceutical composition of the present invention in total of the egg shell membrane ingredient may be 18 mg to 48,000 mg per day for example, and more preferably 35 mg to 3,500 mg.

In addition, the composition for external use containing approximately 1 to 400 mg/ml (0.1 to 40%) of the egg shell membrane ingredient may be painted 1 to several times per day, which will vary in accordance with the area, part and the like of the skin to be applied. An applying method is not limited to a painting, but examples thereof may include an atomizing in case where the composition is liquid and a pasting in case where the composition is in the form of a film, which may be appropriately selected.

Because the agent for activating a sirtuin gene of the present invention has an extremely high level of safety but no side effects, the intake or dose is allowed to exceed the range as described above, provided that another ingredient is appropriately selected when the compound is produced.

(Composition for External Use)

Blending the agent for activating a sirtuin gene of the present invention with a commonly used and publicly known ingredient can prepare a topically applied agent in various dosage forms such as a liquid formulation, solid formulation and semi-solid formulation, in accordance with the intended use. In the composition for external use of the present invention, the agent for activating a sirtuin gene of the present invention and an excipient, along with an active ingredient for beauty or medicine, a fragrance ingredient (such as perfume) and a coloring agent may be used, for example. Examples of another active ingredient include antiflash agent, antiinflammatory agent, melanin production inhibitor, melanin reductant, decolorizer, melanin discharge accelerant, cell activator, antioxidant, oxidation inhibitor, keratin lysing or separating agent, sebum suppressant, moisturizer, emollient, sebum secretion suppressant or accelerant, ultraviolet light absorber, antiperspirant, blood circulation promoter, keratin remover or softening agent, whitening agent, antiallergic agent, steroid hormone, immunosuppressive agent, antibiotic and the like.

For example, blending the agent for activating a sirtuin gene of the present invention with one or more ingredients of hydrocarbon (such as vaseline), higher fatty acid lower alkyl ester (such as stearyl alcohol and isopropyl myristate), animal fat and oil (such as lanolin), polyhydric alcohol (such as glycerin), surfactant (such as glycerin fatty acid ester, polyethylene glycol monostearate), inorganic salt, wax, resin, water, preservative (such as methyl parahydroxybenzoate and butyl parahydroxybenzoate), peptide (such as acetyl hexapeptide-3, palmitoyl pentapeptide-4 (Matrixyl)), sodium acetylated hyaluronate, caprylyl glycol and the like may produce a pharmaceutical composition or cosmetic product for enhancing the moisture level and/or the elasticity of the skin or for improving the general condition.

The composition for external use of the present invention, when being used as an aqueous composition, preferably contains a moisturizing ingredient and/or increasing viscosity ingredient. Examples of the base moisturizing ingredient include glycerin, diglycerin, polyglycerin, propylene glycol, dipropylene glycol, 1-3 butylene glycol, hexylene glycol, maltitol, mannitol, sorbitol, xylitol, trehalose, sodium pyrrolidone carboxylate, sodium polyglutamate, sodium lactate, sodium polylactate, polyethylene glycol, saccharide, methylglucoside and the like. Examples of the increasing viscosity ingredient include sodium hyaluronate, sodium dermatan sulfate, dextrin, sodium arginine, carrageenan, xanthan gum, cornstarch, tragacanth rubber, casein, polyvinyl alcohol, polyvinylpyrrolidone, methylcellulose, hydroxypropyl cellulose, xylan, mannan, galactan, pectin, extensin, arabic gum, pullulan, sodium polyacrylate, carboxyvinyl polymer, clay mineral and the like. In addition, 2-methacryloyloxyethylphosphorylcholine (MPC) polymer is preferable because it can provide a skin-like environment for a fibroblast. 1-3 Butylene glycol, a moisturizing ingredient of the base, is preferably contained in compositions for external use; however, there are cases that are not preferable due to the association with allergy. Therefore, the ingredients are arbitrarily selected depending on symptoms.

(Composition for Oral Use)

The agent for activating a sirtuin gene of the present invention may be prepared into a composition for oral use in the form of a tablet, powder, granule, capsule, liquid and the like. Various ingredients and producing methods for producing a composition for oral use in various types of dosage form are publicly known in the field of production of medicine, cosmetic product and the like, and a person skilled in the art can appropriately select it in accordance with the situation.

The composition for oral use of the embodiment preferably contains an excipient or a diluent base along with at least one of (1) a health builder (for example, vitamin, β-carotene, royal jelly) and (2) any pharmaceutical ingredient available for use in combination (for example, antiinflammatory agent).

The type of vitamin contained in the composition for oral use of the embodiment is not limited in particular, but may be any vitamin as far as humans or mammals can take in. Examples of the vitamin may include a fat soluble vitamin such as vitamin A, vitamin D, vitamin E, vitamin F and vitamin K, and a water soluble vitamin such as vitamin B, vitamin C, vitamin H and vitamin L. The tablet of the embodiment may contain one or more of these vitamins. The content of β-carotene and vitamins may be appropriately determined in accordance with the appropriate amount of each vitamin the subject such as a human takes in. According to the recommendation by the American Heart Association, “healthy people should consume vitamins/minerals, anti-oxidative supplements from food not as supplement intake”. Therefore, preferably the vitamin preparation is not contained in the compositions for oral use, but can be contained when needed, regarding vitamin preparations contained in a composition for oral use.

It is particularly preferable that the composition for oral use of the present invention be in the form of a tablet from the point of view of uniformly containing the egg shell membrane at high density, not undergoing the deformation or disintegration during preservation, circulation and dosing of the composition for example, having an excellent suitability to handling, and being easily dosed orally. As an example of the pharmaceutical composition using the agent for activating a sirtuin gene of the present invention, the tablet is described below.

The content of the egg shell membrane ingredient in the fine powder form contained in the tablet of the embodiment is not limited in particular. However, from the point of view that the granulation and compression are smoothly performed, a sirtuin gene activation effect is enhanced when the tablet is orally taken (dosed), the ability to reduce or scavenge the active oxygen generated in vivo is increased, and the like, the egg shell membrane ingredient is preferably contained in a ratio of 5 to 40% by mass with respect to the total mass of the tablet, and more preferably contained in a ratio of 10 to 25% by mass with respect thereto.

When the content of the egg shell membrane ingredient is 5% by mass or more, there is no need to take in a lot of tablets. On the other hand, when the content of the egg shell membrane in the tablet is 40% by mass or less, the granulation and compression are so easy that the tablet is easily produced.

To the tablet of the embodiment, as various additives to form a tablet, an excipient or a diluent base along with a bonding agent, a disintegrating agent, a lubricant, another nutrient and the like may be appropriately added.

As the excipient or the diluent base for the tablet, at least one of chemically modified starch and lactose is preferably used. The content of the excipient or the diluent base is, from the point of view of the formativeness or making easy to take, preferably a 0.5 to 3-fold amount by mass of the egg shell membrane ingredient, and more preferably a 1 to 2.5-fold amount by mass thereof. Examples of the chemically modified starch may include dextrin such as roasted dextrin (such as white dextrin and yellow dextrin), oxidized starch (such as starch oxidized by hypochlorous acid) and less viscous modified starch (such as acid dipped starch and enzymatically treated starch), and one or more thereof may be used. When as the excipient or the diluent base, the chemically modified starch (in particular, “Waxy a” and “Pine Fiber”) and lactose are used in combination, the use ratio of the chemically modified starch:lactose (mass ratio) is preferably 1:5 to 5:1, and more preferably 1:3 to 3:1.

As the bonding agent, a publicly known bonding agent may be appropriately used, and examples thereof may include starch paste, arabic gum, hydroxypropyl cellulose and the like.

As the disintegrating agent, a publicly known disintegrating agent may be appropriately used, celluloses and the like may be used for example. Note that the starch has a function as the disintegrating agent.

As the lubricant, a publicly known lubricant may be appropriately used, and examples thereof may include wax such as magnesium stearate and/or sucrose fatty acid ester, and talc, vitamin C and the like.

Furthermore, it is particularly preferable for the tablet of the embodiment to contain an egg shell calcium as a hardness improver for raising the hardness of the tablet and preventing the tablet from being deformed and scratched, with the result that the suitability to handling of the tablet during packaging, preservation and circulation of the tablet for example is enhanced, and the uptake is improved. The egg shell calcium is a fine powder derived from a pulverized and dried shell of a bird egg such as a chicken egg, but in the tablet of the embodiment, any egg shell calcium a person can take in may be used. As the egg shell calcium, for example, a conventionally commercially available product name “Calhope” made by Kewpie Corporation and egg shell calcium made by Taiyo International, Inc. may be directly used. The content of the egg shell calcium contained in the tablet is preferably 5 to 20% by mass with respect to the total mass of the tablet, and more preferably 8 to 15% by mass with respect thereto.

The tablet of the embodiment is preferably coated with a coating membrane for the purpose of the prevention from the deterioration and decomposition of the ingredient contained in the tablet and the improvement of the tablet surface tolerance to scratches. The coating membrane may be formed of the same membrane forming material as conventionally used one as a coating membrane of a tablet. As the membrane forming material, a product name “Shellac” (track 30) made by Gifu Shellac Manufacturing Co., Ltd. may be used for example, but the membrane forming material is not limited thereto in particular.

In addition, the tablet of the embodiment is preferably coated with a sugar coating in order to be easily taken orally. Furthermore, the tablet of the embodiment may be colored or glazed after being colored, if necessary.

The size of the tablet of the embodiment is not limited in particular but may be determined appropriately. In general, from the point of view of the suitability to handling and the easiness of dosing, it is preferable that the tablet be rounded or ovalized with the diameter being approximately 7 to 10 mm.

Furthermore, for example, it is preferable for the tablet of the embodiment that the weight of one tablet be approximately 350 to 600 mg, and it is preferable that the amount of the egg shell membrane ingredient contained in one tablet be approximately 18 to 240 mg, and more preferable that the amount thereof be 35 to 150 mg. For example, when approximately 18 to 240 mg of the egg shell membrane ingredient is assumed to be contained in one tablet of the embodiment, 1 to 200 tablets per day (total of 18 to 48,000 mg of the egg shell membrane ingredient per day) may be taken by or administered to an adult.

The tablet of the embodiment may be appropriately produced by a publicly known tablet producing method from a raw material for compression at least containing the egg shell membrane-containing fine powder of the embodiment. Specifically, the tablet of the embodiment may be produced by at least undergoing an uncoated tablet forming step (compressing step) of compressing the raw material for compression to form an uncoated tablet. In addition to the uncoated tablet forming step, a granulating step, protective coating applying step, sugar coating applying step and the like may be performed, and further a coloring, glazing and the like may be applied. The tablet of the embodiment obtained in this way is screened, measured, packaged and the like before being shipped.

(Food Additive)

The agent for activating a sirtuin gene of the present invention may be used alone or in combination with any physiologically acceptable ingredient such as another food additive, as a food additive to be added in a food such as a confectionary, health food, preserved food and processed food. The food additive of the present invention may be added in various foods by a publicly known method in the technical field, for the purpose of the activation of a sirtuin gene. For example, the application of the egg shell membrane to a food is suggested in JP 3862600 B1 and JP 2009-165421 A, which disclose a tablet, confectionery and the like containing an egg shell membrane pulverized into the fine powder form. As an egg shell membrane powder used in the tablet or confectionery disclosed in these documents, the food additive containing the agent for activating a sirtuin gene of the present invention may be used.

Note that “food” used herein is not limited for humans but includes a feed for mammals such as dog and cat reared as a companion or domestic animal. In addition, in the concept of “food”, a normal food along with a beverage, so-called supplement, health food, enteral nutritional food, food for special use, nutritional and functional food, food for specified health use and the like are included.

### EXAMPLES

Hereinafter, a description is made of some examples of the present invention, but the present invention is not limited to only the following examples.

1. Production of Agent for External Use Containing Egg Shell Membrane Hydrolyzate

As an alkaline hydrolyzed egg shell membrane (hereinafter referred to as “ASESM”), a product name “EM PROTEIN-P” available from Kewpie Corporation, Tokyo, Japan was used. It was found that the relative molecular weight of the main part of this ASESM measured by a size exclusion chromatography (gel filtration) was approximately 12 to 14 kDa (Ohto-Fujita et al, Cell Tissue Res. 2011 July; 345(1): 177-190).

A solution (lotion) containing 10% (W/V) ASESM was produced with the use of an aqueous solution of 7% (V/V) butylene glycol, 1% (V/V) pentylene glycol, 4% (V/V) glycerin and 0.2% (V/V) phenoxyethanol as a basis.

2. Effect of ASESM Agent for External Use on Expression of Sirtuin and Extracellular Matrix (ECM) Related Genes in Back of Hairless Mouse

Animals used were hairless mice (Hos: HR-1, 6 weeks of age, male) (a control group: n=9, an ASESM treated group: n=9). To the dorsal skin of the ASESM treated group, 10% (W/V) ASESM solution produced as described above was applied externally (locally) for 10 days (40 it/application×2 every day) or for 14 days (20 it/application×1 on the first day and second day, 40 it/application×2 on the third day to ninth day, and 40 it/application×1 on the tenth day to fourteenth day). In the same way, to the control group, the basis solution not containing the ASESM was applied.

A quantitative real-time polymerase chain reaction (quantitative real-time PCR) analysis was performed as follows. A skin sample was collected from each mouse and pulverized in liquid nitrogen. After the whole skin tissue was homogenized, total RNA was isolated with the use of a product name “TRIzol (registered trademark) Reagent”. The total RNA (200 ng) was used in a cDNA synthesis in which a product name “Takara PrimeScript RTR reagent kit” was used. The real-time PCR was performed with the use of a product name “SYBRR Premix Ex Taq™ II (Takara) on Thermal Cycler Dice Real Time System” (Takara). As a primer, primers designed to amplify genes coding type I collagen, type III collagen, type IV collagen, MMP2, matrix metalloproteinase 3 (MMP3), heat-shock protein 47 (Hsp47), elastin, decorin, hyaluronan synthetase 2 (Has2), transforming growth factor β1 (TGF-β1), transforming growth factor β (TGF-β3), sirtuin 1 (SIRT1), sirtuin 2 (SIRT2), sirtuin 3 (SIRT3), sirtuin 4 (SIRT4), sirtuin 5 (SIRT5), sirtuin 6 (SIRT6) and sirtuin 7 (SIRT7) were used. As an internal standard, glyceraldehyde-3-phosphate dehydrogenase (GAPDH) mRNA that was a housekeeping gene was amplified in the same way. The PCR cycle was programed as follows: a first denaturation at 95° C. for 30 seconds, followed by 40 cycles of an amplification (including a denaturation at 95° C. for 5 seconds, and an annealing and elongation at 60° C. for 1 minute). The primers used are collectively shown in the following table.

TABLE 1

| | | GENE | | | | AMPLICON |
|---|---|---|---|---|---|---|
| SEQUENCE | TARGET FOR | ACCESSION | PRIMER | SEQUENCE | | SIZE |
| NUMBER | AMPLIFICATION | NUMBER | NAME | (5′ → 3′) | LENGTH | (bp) |
| 1 | Mus Musculus SURTUIN 1 | NM_019812 | Mm_Sirt1-F-2 | TCCTGACTTCAGAT | 24 | 172 |
| | | | | CAAGAGACGG | | |
| 2 | | | Mm_Sirt1-R-2 | GTGACACAGAGACG | 20 | |
| | | | | GCTGGA | | |
| 3 | Mus Musculus SURTUIN 2 | NM_022432 | Mm_Sirt2-F | CGGGCAGTTCAAGC | 19 | 98 |
| | | | | CAACC | | |
| 4 | | NM_001122765.1 | Mm_Sirt2-R | CCAGCGTGTCTATG | 21 | |
| | | | | TTCTGCG | | |
| 5 | Mus Musculus SURTUIN 3 | NM_022433 | Mm_Sirt3-F | GACGGGCTTGAGAG | 21 | 135 |
| | | | | AGCATCT | | |
| 6 | | NM_001127351.1 | Mm_Sirt3-R | GTCCGCCATCACAT | 19 | |
| | | | | CAGCC | | |
| 7 | Mus Musculus SURTUIN 4 | NM_001167691 | Mm_Sirt4-F | CCAACCCAACCCAG | 20 | 161 |
| | | | | CACACT | | |
| 8 | | NM_133760 | Mm_Sirt4-R | CACAGTTCAGGCAC | 22 | |
| | | | | AGGACTCT | | |
| 9 | Mus Musculus SURTUIN 5 | NM_178848 | Mm_Sirt5-F | TGTGACCTGTGTCT | 22 | 176 |
| | | | | AGTGGTGG | | |
| 10 | | | Mm_Sirt5-R | GCTTCAGGAAGAGT | 23 | |
| | | | | TTTCCCACA | | |
| 11 | Mus Musculus SURTUIN 6 | NM_181586 | Mm_Sirt6-F | GACTTCGGGCCTGT | 20 | 116 |
| | | | | AGAGGG | | |
| 12 | | NM_001163430 | Mm_Sirt6-R | GACAGACAGGTCTG | 20 | |
| | | | | CGGTCC | | |
| 13 | Mus Musculus SURTUIN 7 | NM_153056 | Mm_Sirt7-F | GCAGTGGACCCCGA | 19 | 130 |
| | | | | AGGAT | | |
| 14 | | | Mm_Sirt7-R | GAAGATTGGATCCT | 21 | |
| | | | | GCCACCG | | |
| 15 | Mus Musculus | NM_008084 | mGAPDH-Left | AGCTTGTCATCAAC | 20 | 62 |
| | GLYCERALDEHYDE-3- | | | GGGAAG | | |
| 16 | PHOSPHATE DEHYDROGENASE | | mGAPDH-Right | TTTGATGTTAGTGG | 21 | |
| | (Gapdh) | | | GGTCTCG | | |
| 17 | Mus Musculus TYPE I alpha | NM_007742 | Mouse Col 1a1 left | CATGTTCAGCTTTG | 21 | 94 |
| | 1 COLLAGEN (Col lal) | | | TGGACCT | | |
| 18 | | | Mouse Col 1a1 right | GCAGCTGACTTCAG | 20 | |
| | | | | GGATGT | | |
| 19 | Mus Musculus TYPE III | NM_009930 | Mouse Col 3a1 left | TCCCCTGGAATCTG | 20 | 63 |
| | alpha 1 COLLAGEN (Col3a1) | | | TGAATC | | |
| 20 | | | Mouse Col 3a1 right | TGAGTCGAATTGGG | 20 | |
| | | | | GAGAAT | | |
| 21 | Mus Musculus TYPE IV | NM_009931 | Mouse Col 4a1 left | TTAAAGGACTCCAG | 21 | 61 |
| | alpha 1 COLLAGEN (Col4a1) | | | GGACCAC | | |
| 22 | | | Mouse Col 4a1 right | CCCACTGAGCCTGT | 19 | |
| | | | | CACAC | | |
| 23 | Mus Musculus MATRIX | NM_008610 | Mouse MMP-2-2 left | GGAGAAGGCTGTGT | 20 | 90 |
| | METALLOPROTEINASE 2 (Mmp2) | | | TCTTCG | | |
| 24 | | | Mouse MMP-2-2 right | AGGCTGGTCAGTGG | 18 | |
| | | | | CTTG | | |
| 25 | Mus Musculus MATRIX | NM_010809 | Mouse MMP-3 left | TTGTTCTTTGATGC | 21 | 74 |
| | METALLOPROTEINASE 3 (Mmp3) | | | AGTCAGC | | |
| 26 | | | Mouse MMP-3 right | GATTTGCGCCAAAA | 18 | |
| | | | | GTGC | | |
| 27 | Mus Musculus Hsp47, ALIAS | NM_009825 | Mouse Hsp47 left | GCGGACTGCCTGGT | 18 | 76 |
| | SERINE (OR CYSTEINE) | | | AAAC | | |
| 28 | PEPTIDASE INHIBITOR, clade | | | GAAGGAGAGAGCGC | 18 | |
| | H, member 1 (Serpinh 1) | | | ATGG | | |
| | transcript variant 1 | | | | | |
| 29 | Mus Musculus ELASTIN (Eln) | NM_007925 | Mm_Eln-F | CTCCTCCATCCCGC | 18 | 174 |
| | | | | GCAG | | |
| 30 | | | Mm_Eln-R | TCCCAGAAGTCCGG | 19 | |
| | | | | CACCT | | |
| 31 | Mus Musculus DECORIN | NM_007833 | mouse decorin left | GAGGAGAAGTGAGG | 19 | 96 |
| | (Dcn), transcript | | | GGAGA | | |
| 32 | variant 2 | | mouse decorin right | CATGATTATCTCAT | 27 | |
| | | | | GTATTTTCACGAC | | |
| 33 | Mus Musculus HYALURONAN | NM_008216 | mouse Has2 left | GGCGGAGGACGAGT | 19 | 62 |
| | SYNTHETASE 2 (Has2) | | | CTATG | | |
| 34 | | | mouse Has2 right | ACACATAGAAACCT | 24 | |
| | | | | CTCACAATGC | | |
| 35 | Mus Musculus TRANSFORMING | NM_011577 | Mm_Tgfb1-F | GGATACCAACTATT | 24 | 62 |
| | GROWTH FACTOR β1 (Tgfb1) | | | GCTTCAGCTC | | |
| 36 | | | Mm_Tgfb1-R | GTGTCCAGGCTCCA | 23 | |
| | | | | AATATAGGG | | |
| 37 | Mus Musculus TRANSFORMING | NM_009368 | Mm_Tgfb3-F | CGGATGAGCACATA | 21 | 138 |
| | GROWTH FACTOR β3 (Tgfb3) | | | GCCAAGC | | |
| 38 | | | Mm_Tgfb3-R | CCAGACCCAAGTTG | 22 | |
| | | | | GACTCTCT | | |
| 39 | SMPD1 | NM_011421 | mouse SMPD1 left | TGCTGAGAATCGAG | 20 | 122 |
| | | | | GAGACA | | |
| 40 | | | mouse SMPD1 right | GACCGGCCAGAGTG | 19 | |
| | | | | TTTTC | | |
| 41 | AQP-3 | NM_016689 | mouse AQP-3 left | CTGGGGACCCTCAT | 18 | 88 |
| | | | | CCTT | | |
| 42 | | | mouse AQP-3 right | TGGTGAGGAAGCCA | 18 | |
| | | | | CCAT | | |
| 43 | MMP-3 | NM_010809 | mouse MMP-3 left | TTGTTCTTTGATGC | 21 | 74 |
| | | | | AGTCAGC | | |
| 44 | | | mouse MMP-3 right | GATTTGCGCCAAAA | 18 | |
| | | | | GTGC | | |
| 45 | Has-1 | NM_008215 | Mouse Has1 left | AAAGAGAACAAGAC | 24 | 74 |
| | | | | GGAGAAGAGA | | |
| 46 | | | Mouse Has1 right | CTGAGGGCTTTGGC | 18 | |
| | | | | ATGT | | |
| 47 | Sptlc-1 | NM_009269 | mouse Sptlc1 left | GGTGCTGGTGGAGA | 18 | 68 |
| | | | | TGGT | | |
| 48 | | | mouse Sptlc1 right | GGATTCCTTCCAAA | 23 | |
| | | | | ATAAGATGG | | |

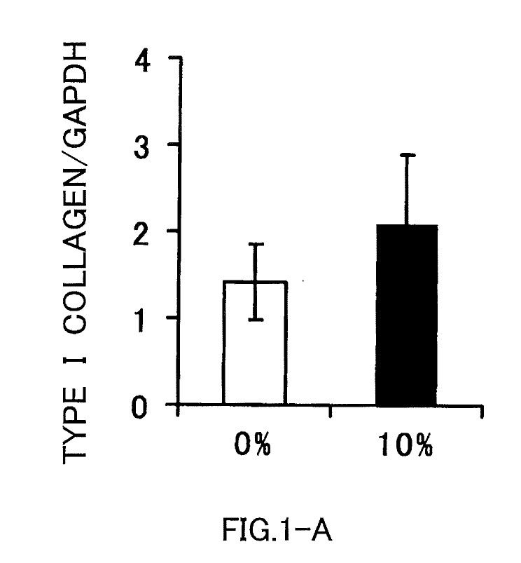

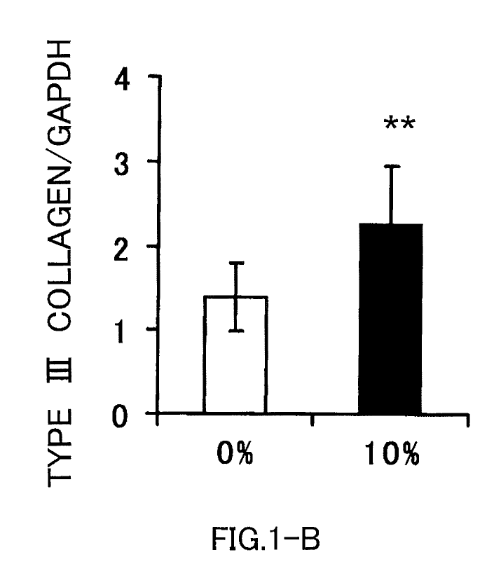

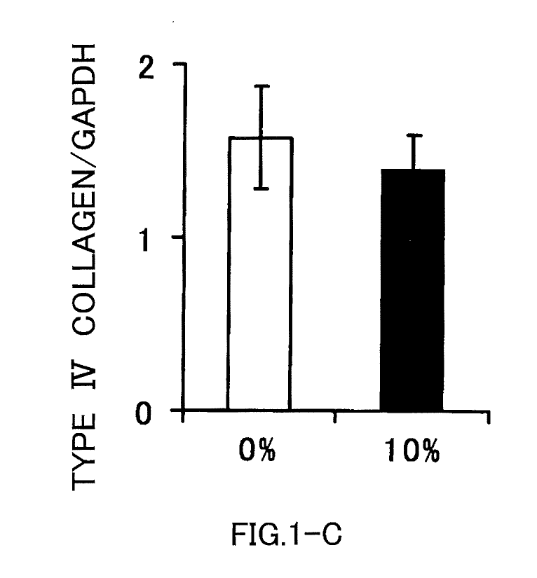

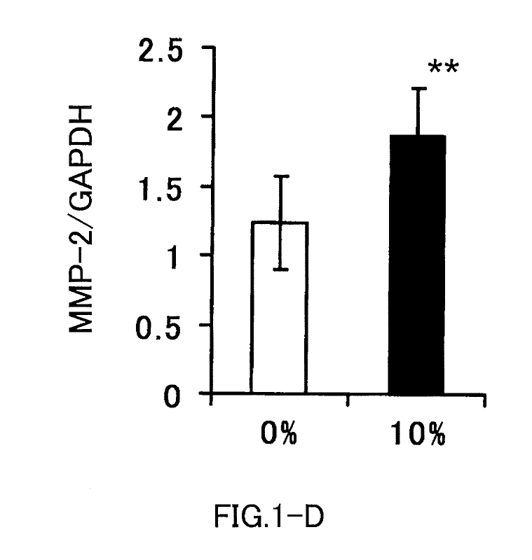

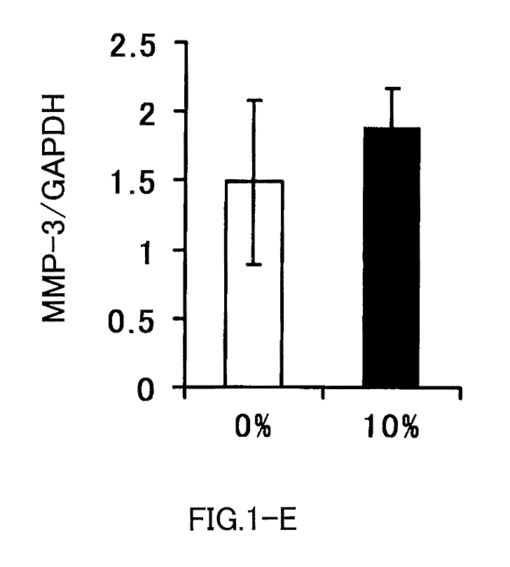

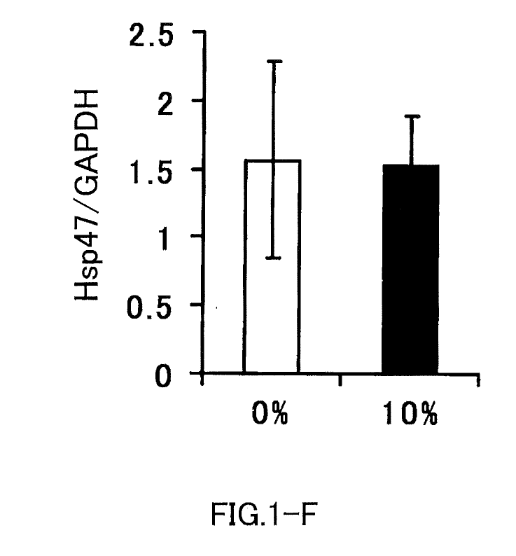

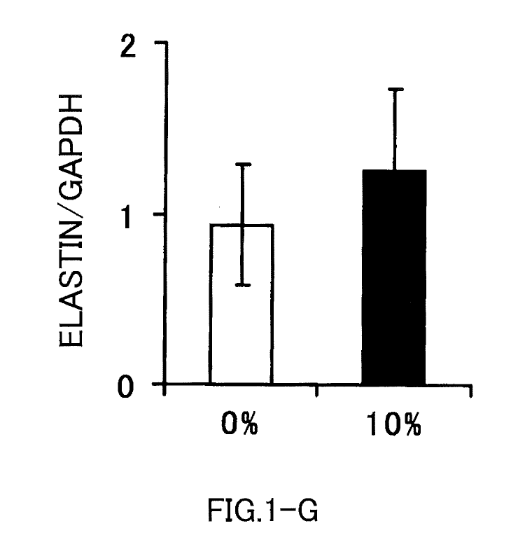

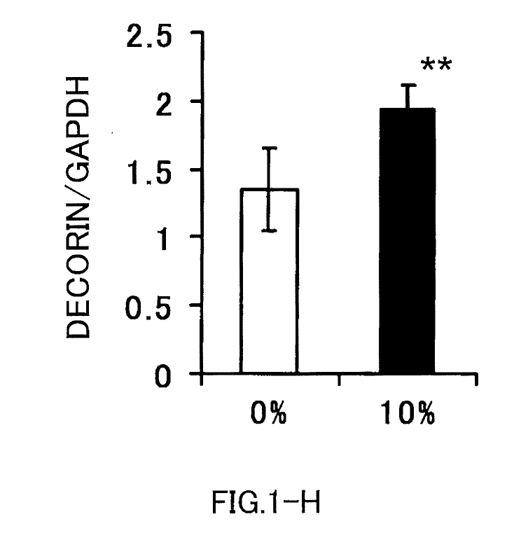

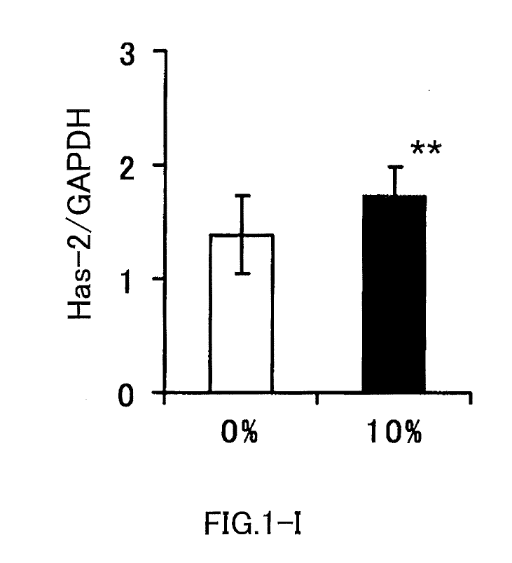

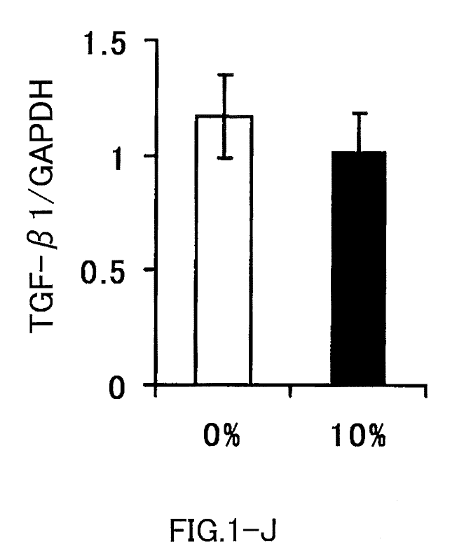

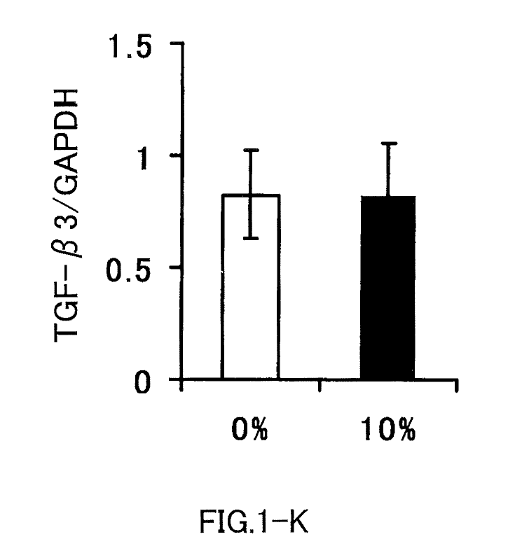

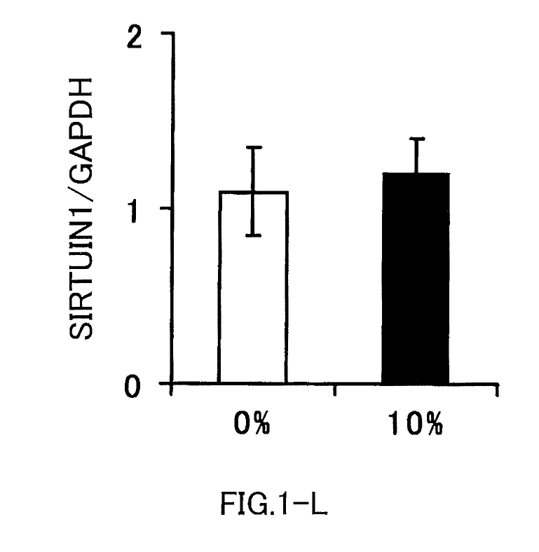

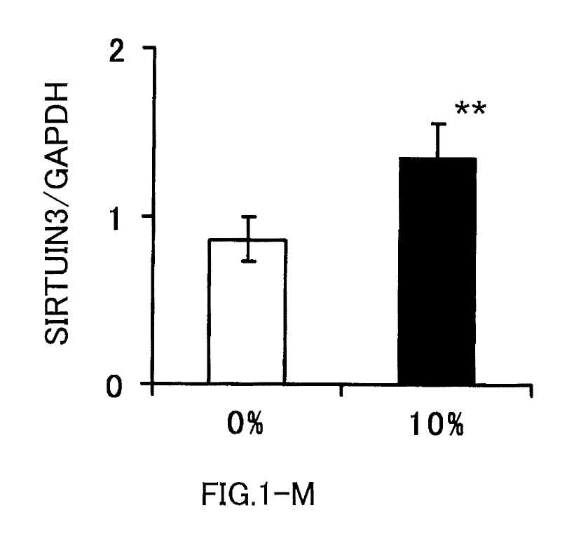

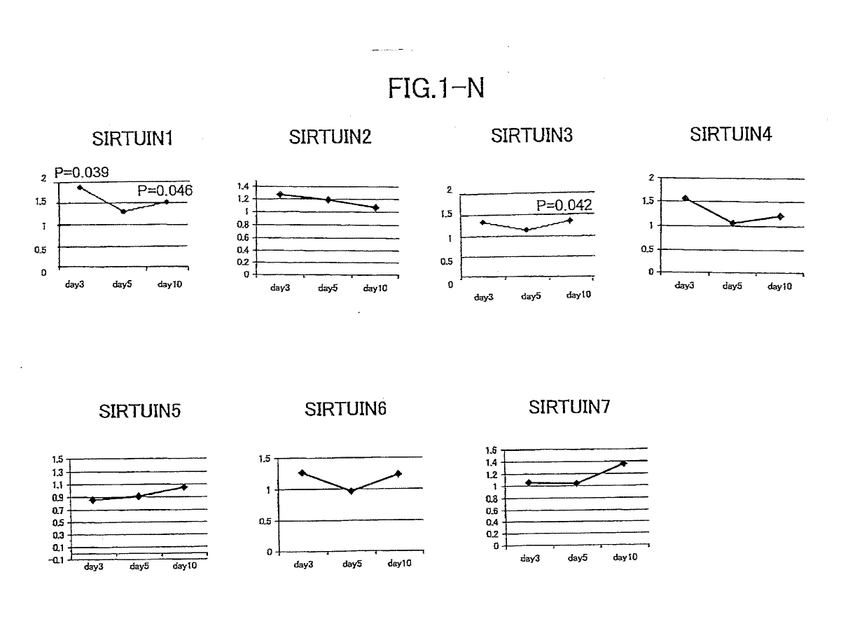

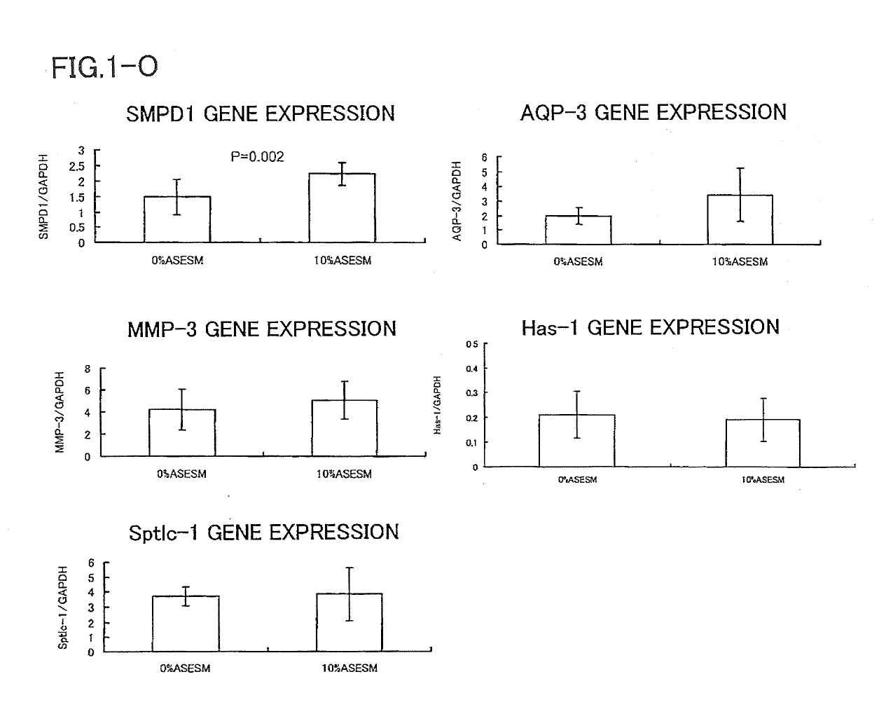

Each gene expression was measured as the ratio to the gene expression of GAPDH. The result is shown in FIG. 1-A to 1-M and table 2.

TABLE 2

| | APPLICATION OF 10% | APPLICATION OF 10% |
|---|---|---|
| | ASESM TO SKIN FOR | ASESM TO SKIN FOR |
| | 10 DAYS | 14 DAYS |
| Sirt 1 | P = 0.321 ↑ 1.10-FOLD | ↑ 8.7-FOLD |
| Sirt 2 | | ↑ 6.7-FOLD |
| Sirt 3 | P = 0.00005 ↑ 1.55-FOLD | ↑ 3.7-FOLD |
| Sirt 4 | | ↑ 9.2-FOLD |
| Sirt 5 | | ↑ 8.6-FOLD |
| Sirt 6 | | ↑ 11.3-FOLD |
| Sirt 7 | | ↑ 9.9-FOLD |

Basically in the same way as described above, to the dorsal skin of hairless mice (6 weeks of age, male, Hos: HR-11), 1% (W/V) ASESM solution was painted for 3, 5 or 10 days, and the expression of the sirtuin genes in the epidermis was examined. The result is shown in FIG. 1-N as the ratio to the value from the control group to which 0% ASESM solution was used.

Furthermore, to the dorsal skin of the hairless mice (female, Kud: HR-), 20 μl of 10% (W/V) ASESM solution or 0% ASESM solution per cm² was painted for 5 days, and basically in the same way as described above, the expression of genes for SMPD1, aquaporin (AQP-3), MMP3, Has1 and sptic-1 that were expressed in the epidermis was examined. The result is shown in FIG. 1-O. The SMPD-1 gene expression was significantly increased, and the aquaporin-3 gene expression was also increased. On the other hand, the gene expression of MMP3, Has1 and sptic-1 was not changed. SMPD-1 is sphingomyelinase that is an enzyme involved in a ceramide synthesis. By the increase of this enzyme, it may be estimated that a ceramide synthetic pathway is stimulated. Because the aquaporin is a water channel that is known to regulate intracellular water not only in the skin but also in the kidney, when the egg shell membrane is taken, it may be hoped that the aquaporin will be effective in the improvement of a lifestyle-related disease such as diabetes.

Thus, in the dorsal skin of the ASESM treated group, the ASESM affected the cell so that the expression of various kinds of sirtuin genes was regulated: the painting with 1% ASESM for 10 days significantly increased the mRNAs of SIRT1 and SIRT3, and the painting with 10% ASESM for 10 days significantly increased the mRNA of SIRT3 in particular. In addition, it was observed that the painting with 10% ASESM for 5 days significantly increased the mRNA of SMPD 1, the mRNA of type III collagen and decorin that were main extracellular matrices (ECM), and the mRNA of MMP2. It was observed that the painting with 10% ASESM for 14 days further markedly increased the mRNA expression of SIRT1 to SIRT7, and increased the mRNA expression of SIRT2 and SIRT4.

3. Effect of ASESM Agent for External Use on Water of Skin of Woman

In order that the effect of the ASESM agent for external use might be evaluated, a double-blind experiment was performed in which a placebo was used as a control. In one individual, the left or right arm (one arm) was not painted randomly. Thirty woman volunteers as subjects (20 to 65 years old, average age: 36.9±13.2 years old) were divide into a control group of 15 people (average age: 35.4±12.7 years old) and an ASESM group of 15 people (average age: 38.3±14.0 years old) at random. In the ASESM group, both moisturizing lotion and cream containing 1% (W/V) ASESM were applied to the front and upper arms twice a day in a predetermined way. In the control group, the same lotion and cream except that the ASESM was not contained were applied in the same way. Prior to the experiment, and 2 weeks and 4 weeks after the experiment was started, the water of the skin inside and outside the upper arm and inside and outside the front arm for each subject was measured with the use of a skin water measuring instrument “Corneometer (registered trademark) CM825, Courage+Khazaka”. In order that a reliable measurement value might be obtained, the subject was acclimatized before the experiment for 15 minutes in a controlled room (22±2° C., 50±10% RH). As the result of the water measurement, no difference was found between the left and right arms. Accordingly, a significance test for the change of the individual initial value was performed in the presence or absence of the ASESM.

A composition of and a method for producing the lotion and cream were as follows.

TABLE 3

| | | | 0% ASESM | |
|---|---|---|---|---|
| | | 1% ASESM | LOTION FOR | |
| | | LOTION FOR | CONTROL | |
| | | EXPERIMENTAL | GROUP | |
| | | GROUP | BLENDING | |
| BLENDING | | BLENDING | QUANTITY | RAW MATERIAL |
| ORDER | INDICATION NAME | QUANTITY (%) | (%) | SOURCE |
| 1 | WATER | 75.59529 | 76.59529 | |
| 2 | BG | 11.29554 | 11.29554 | KYOWA HAKKO KOGYO |
| | | | | CO., LTD. |
| 3 | GLYCERIN | 5.4 | 5.4 | KAO CORPORATION |
| 4 | 1,2-HEXANEDIOL | 2.5 | 2.5 | OSAKA ORGANIC |
| | | | | CHEMICAL INDUSTRY |
| | | | | LTD. |
| 5 | PEG-75 | 1.5 | 1.5 | |
| 6 | HYDROLYZED EGG | 1 | 0 | KEWPIE CORPORATION |
| | SHELL MEMBRANE | | | |
| 7 | LYSOLECITHIN | 0.2 | 0.2 | KEWPIE CORPORATION |
| 8 | GLUCAN | 0.6 | 0.6 | NIKKO CHEMICALS |
| | OLIGOSACCHARIDE | | | CO., LTD. |
| 9 | ROSE EXTRACT | 0.00025 | 0.00025 | YAMAMOTO PERFUMERY |
| | | | | CO., LTD. |
| 10 | HYDROLYZED | 0.0005 | 0.0005 | ICHIMARU PHARCOS |
| | COLLAGEN | | | CO., LTD. |
| 11 | SORBITOL | 0.7 | 0.7 | TOWA-KASEI CO., |
| | | | | LTD. |
| 12 | PHENOXYETHANOL | 0.2 | 0.2 | Lipotec S.A. |
| 13 | HYDROLYZED YEAST | 0.0016 | 0.0016 | MARUZEN |
| | EXTRACT | | | PHARMACEUTICALS |
| | | | | CO., LTD. |
| 14 | BROWN ALGAE | 0.00306 | 0.00306 | MARUZEN |
| | EXTRACT | | | PHARMACEUTICALS |
| | | | | CO., LTD. |
| 15 | ALOE EXTRACT | 0.0007 | 0.0007 | KOEI KOGYO CO., |
| | | | | LTD. |
| 16 | PANAX GINSENG | 0.00126 | 0.00126 | MARUZEN |
| | ROOT EXTRACT | | | PHARMACEUTICALS |
| | | | | CO., LTD. |
| 17 | SCUTELLARIA | 0.0018 | 0.0018 | MARUZEN |
| | BAICALENSIS ROOT | | | PHARMACEUTICALS |
| | EXTRACT | | | CO., LTD. |
| 18 | ARNICA EXTRACT | 0.10002 | 0.10002 | ICHIMARU PHARCOS |
| | | | | CO., LTD. |
| 19 | CUCUMBER EXTRACT | 0.10002 | 0.10002 | ICHIMARU PHARCOS |
| | | | | CO., LTD. |
| 20 | HEDERA HELIX | 0.10002 | 0.10002 | ICHIMARU PHARCOS |
| | EXTRACT | | | CO., LTD. |
| 21 | SAMBUCUS NIGRA | 0.10002 | 0.10002 | ICHIMARU PHARCOS |
| | FLOWER EXTRACT | | | CO., LTD. |
| 22 | MALVA SYLVESTRIS | 0.09996 | 0.09996 | ICHIMARU PHARCOS |
| | MALLOW EXTRACT | | | CO., LTD. |
| 23 | PARIETARIA | 0.09996 | 0.09996 | ICHIMARU PHARCOS |
| | OFFICINALIS | | | CO., LTD. |
| | EXTRACT | | | |
| 24 | DPG | 0.4 | 0.4 | KEWPIE CORPORATION |
| | TOTAL | 100 | 100 | |

TABLE 4

| | | 1% ASESM | | |
|---|---|---|---|---|
| | | CREAM FOR | 0% ASESM | |
| | | EXPERIMENTAL | CREAM FOR | |
| | | GROUP | CONTROL GROUP | |
| BLENDING | | BLENDING | BLENDING | SOURCE TO |
| ORDER | INDICATION NAME | QUANTITY (%) | QUANTITY (%) | PURCHASE |
| 1 | WATER | 63.01449 | 64.01449 | |
| 2 | BG | 9.015 | 9.015 | KYOWA HAKKO |
| | | | | KOGYO CO., |
| | | | | LTD. |
| 3 | GLYCERIN | 7 | 7 | KAO |
| | | | | CORPORATION |
| 4 | SQUALANE | 6.775 | 6.775 | KISHIMOTO |
| | | | | SPECIAL LIVER |
| | | | | OIL CO., LTD. |
| 5 | PHYTOSTERYL/ISOSTEARYL/ | 4 | 4 | NIPPON FINE |
| | CETYL/STEARYL/BEHENYL | | | CHEMICAL CO., |
| | DIMER DILINOLEATE | | | LTD. |
| 6 | HYDROGENATED CASTOR | 4 | 4 | NATIONAL |
| | OIL STEARATE | | | MIMATSU |
| | | | | CORPORATION |
| 7 | 1,2-HEXANEDIOL | 2 | 2 | OSAKA ORGANIC |
| | | | | CHEMICAL |
| | | | | INDUSTRY LTD. |
| 8 | HYDROXYETHYL | 1.875 | 1.875 | SEIWA SUPPLY |
| | ACRYLATE/SODIUM. | | | CO., LTD. |
| | ACRYLOYLDIMETHYL | | | |
| | TAURATE COPOLYMER | | | |
| 9 | POLYSORBATE 60 | 0.275 | 0.275 | SEIWA SUPPLY |
| | | | | CO., LTD. |
| 10 | SORBITAN ISOSTEARATE | 0.075 | 0.075 | |
| 11 | XANTHAN GUM | 0.1 | 0.1 | DAINIPPON |
| | | | | SUMITOMO |
| | | | | PHARMA CO., |
| | | | | LTD. |
| 12 | HYDROLYZED EGG SHELL | 1 | 0 | KEWPIE |
| | MEMBRANE | | | CORPORATION |
| 13 | PLATINUM | 0.045-0.055 ppm | 0.045-0.055 ppm | INOVEX- |
| | | | | CORPORATION |
| 14 | GLUCAN OLIGOSACCHARIDE | 0.6 | 0.6 | |
| 15 | HYDROLYZED COLLAGEN | 0.0005 | 0.0005 | ICHIMARU |
| | | | | PHARCOS CO., |
| | | | | LTD. |
| 16 | SODIUM HYALURONATE | 0.05 | 0.05 | KEWPIE |
| | | | | CORPORATION, |
| | | | | KIBUN FOOD |
| | | | | CHEMIFA CO., |
| | | | | LTD. |
| 17 | PHENOXYETHANOL | 0.2 | 0.2 | Lpotec S.A. |
| 18 | ROSE EXTRACT | 0.02 | 0.02 | YAMAMOTO |
| | | | | PERFUMERY |
| | | | | CO., LTD. |
| 19 | POLYSORBATE 80 | 0.00001 | 0.00001 | |
| | TOTAL | 100 | 100 | |

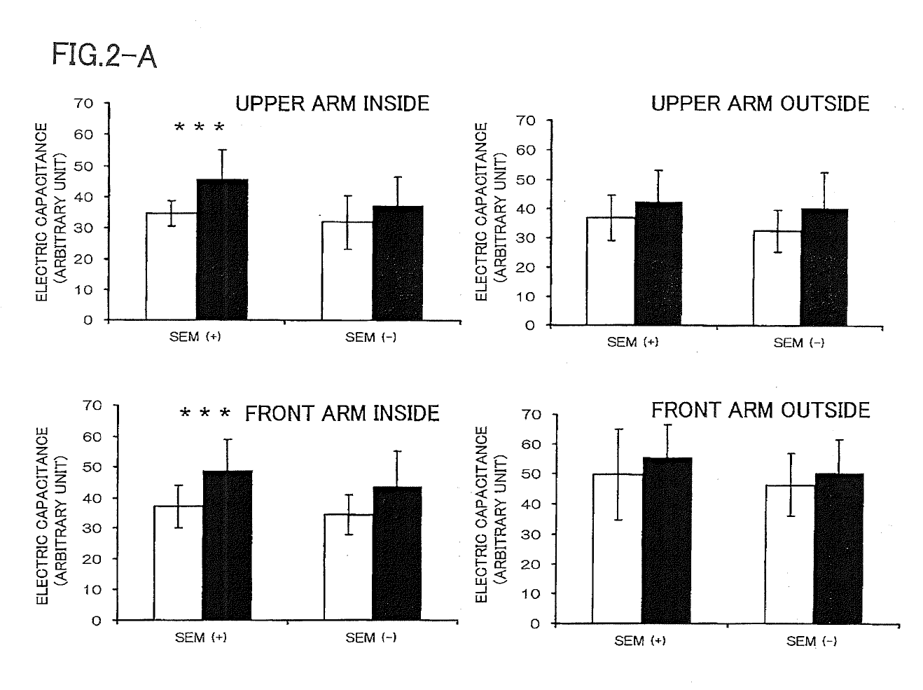

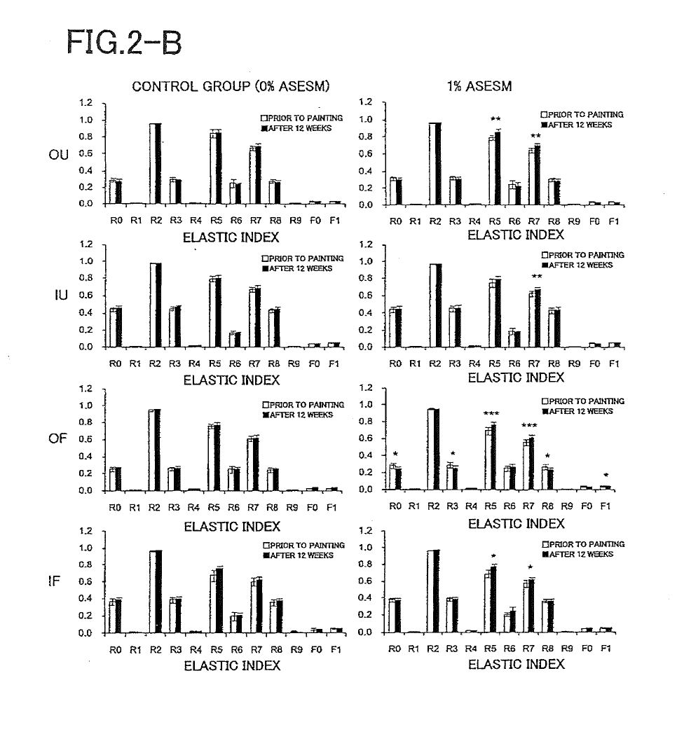

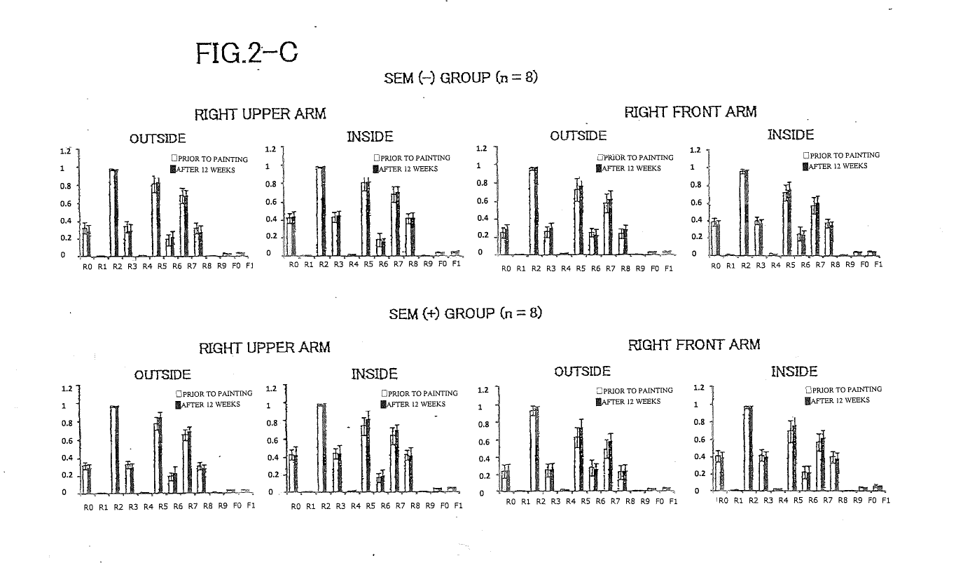

The comparison of the result before the experiment was started with the result 4 weeks after the experiment was started is shown in FIG. 2-A. In the measurement after 4 weeks, the water of the skin inside the upper arm was significantly increased (***p<0.001).

4-1. Effect of ASESM Agent for External Use on Elasticity of Skin of Woman <Left Arm>

In order that the effect of the ASESM agent for external use might be evaluated, a double-blind experiment was performed in which a placebo was used as a control. A subject was the same as the subject who underwent the water measurement. In one individual, the left or right arm (one arm) was not painted randomly. In contrast to the effect on the water, it was found that the presence or absence of the ASESM resulted in the difference of elasticity between the left and right arms. Accordingly, a statistical treatment was performed separately for the left and right arms. A description is made of a left arm group. Fourteen woman volunteers as subjects (22 to 54 years old, average age: 37.1±12.6 years old) were divide into a control group (average age: 37.4±13.5 years old) and an ASESM group (average age: 36.7±12.8 years old) at random. For 12 weeks, in the ASESM group, both moisturizing lotion and cream containing 1% (W/V) ASESM of the same as described in section 3 were applied to the front and upper arms twice a day in a predetermined way. In the control group, the same lotion and cream except that the ASESM was not contained were applied in the same way.

Twelve weeks later, the elasticity of the skin for each subject was measured with the use of a skin viscoelasticity measuring instrument “CutometerR MPA 580, Courage+Khazaka”. The measurement areas were on the front and upper arms. In order that a reliable measurement value might be obtained, the subject was acclimatized before the experiment for 15 minutes in a controlled room (22±2° C., 50±10% RH).

The elastic characteristic was measured by a cutometer under the following conditions: a time/strain mode, an application of 300 mbar constant negative pressure for 3 seconds, followed by a relaxation period for 3 seconds. The cutometer parameters (viscoelastic indices) are as follows: Ue=immediate distention; Uv=delayed distention; Uf=final distention (skin distensibility); Ur=immediate retraction; Ua=final retraction; Ua/Uf=gross-elasticity of the skin, including viscous deformation; Ur/Ue=neto-elasticity of the skin without viscous deformation; Ur/Uf=biological elasticity, i.e., the ratio of immediate retraction to total distension; Uv/Ue=the ratio of viscoelastic to elastic distension; R8=viscopart, i.e., the area under the suction part of the deformation curve; R=residual deformation at the end of measuring cycle (resilient distension);

Elastic Indices

1. R0=Uf=Ue+Uv (maximum value of waveform amplitude) (First max. amplitude)
2. R1=Uf−Ua (re-deformation ability) (First min. amplitude)
3. R2=Ua/Uf (gross elasticity) (Gross-elasticity of the skin, including viscous deformation)
4. R3=last maximum amplitude (Last max. amplitude)
5. R4=last minimum amplitude (Last min. amplitude)
6. R5=Ur/Ue (elasticity) (neto-elasticity of the skin without viscous deformation)
7. R6=Uv/Ue (ratio of plastic range) (the ratio of viscoelastic to elastic distension)
8. R7=Ur/Uf (elasticity) (Bio-logical elasticity)
9. R8=Ua of the first curve (Ua of the first curve)
10. R9=R3−R0 (skin fatigue after successive suction)
11. F0=(integrated value) (surface area)
12. F1=(integrated value) (surface area)

The result is shown in FIG. 2-B. Before and after the outside upper arm (OU), inside upper arm (IU), outside front arm (OF) and inside front arm (IF) were treated for 12 weeks with the composition not containing the ASESM (0% (W/V) ASESM) (the control group, n=7) and the composition containing 1% (W/V) ASESM (the ASESM group, n=7), the elasticity of the skins was measured. In the control group treated with the composition not containing the ASESM, the significant change in 12 elastic indices of the left front arm and upper arm were not observed. On the other hand, in the ASESM group, the significant increase of elasticity was obtained in R7 (8.6% increased) and R5 for OU, IU, OF and IF (*P<0.05, **P<0.01, ***P<0.001). Only for OF, the significant increase in some other indices (R0, R3, R8 and F1) was obtained after 12 weeks (*P<0.05). Note that each value of R0, R1, R3, R4, R8 and R9 is a calculated length (mm), and each value of R2, R5, R6, R7, F0 and F1 is expressed in an arbitrary unit.

4-2. Effect of ASESM Agent for External Use on Elasticity of Skin of Woman <Right Arm>

A description is made of a right arm group. Twenty nine woman volunteers as subjects (22 to 54 years old, average age: 37.1±12.6 years old) were divide into a control group (average age: 37.4±13.5 years old) and an ASESM group (average age: 36.7±12.8 years old) at random. In the ASESM group, both moisturizing lotion and cream containing 1% (W/V) ASESM of the same as described above were applied to the front and upper arms twice a day for 12 weeks in a predetermined way. In the control group, the same lotion and cream except that the ASESM was not contained were applied in the same way.

Prior to the experiment, and 2 weeks, 4 weeks, 8 weeks and 12 weeks after the experiment was started, the elasticity of the skin of the right arm for each subject was measured with the use of a skin viscoelasticity measuring instrument “CutometerR MPA 580, Courage+Khazaka”. The measurement areas were on the front and upper arms. In order that a reliable measurement value might be obtained, the subject was acclimatized before the experiment for 15 minutes in a controlled room (22±2° C., 50±10% RH).

The result is shown in FIG. 2-C and Table 5. The elasticity of the right arm was significantly increased after 12 weeks. Note that in contrast to the left arm, in the right arm, although some indices of elasticity in the control group were increased, the rate of change after 12 weeks was several times higher in the ASESM painted group. It is likely that the difference in response between the right and left arms is because the motion characteristic and use frequency of the right arm that is more dominant in motion in daily living activity are high. Accordingly, a synergistic effect may be expected when the ASESM painting and a mechanical stimulation (such as a massage and exercise) to the skin are performed in parallel.

TABLE 5

| | BEFORE | | AFTER | | | | BEFORE | | AFTER | | |
|---|---|---|---|---|---|---|---|---|---|---|---|
| | PAINTING | 1 SD | 12 WEEKS | 5 SD | P | | PAINTING | 1 SD | 12 WEEKS | 5 SD | P |
| ALL AGES A RIGHT-UPPER-OUTSIDE | | | | | | ALL AGES A RIGHT-UPPER-INSIDE | | | | | |
| R0 | 0.3246 | 0.0645 | 0.2814 | 0.0755 | **↓, −13.5 | R0 | 0.4254 | 0.0568 | 0.4417 | 0.0562 | |
| R1 | 0.0089 | 0.0021 | 0.0095 | 0.0021 | | R1 | 0.0102 | 0.0030 | 0.0101 | 0.0033 | |
| R2 | 0.9716 | 0.0085 | 0.9644 | 0.0115 | *↓, −0.8 | R2 | 0.9754 | 0.0099 | 0.9761 | 0.0106 | |
| R3 | 0.3334 | 0.0648 | 0.2894 | 0.0762 | ***↓, −13.2 | R3 | 0.4356 | 0.0558 | 0.4511 | 0.0556 | |
| R4 | 0.0169 | 0.0057 | 0.0168 | 0.0048 | | R4 | 0.0168 | 0.0062 | 0.0177 | 0.0059 | |
| R5 | 0.8130 | 0.0933 | 0.8268 | 0.0624 | | R5 | 0.8061 | 0.0818 | 0.8133 | 0.0756 | |
| R6 | 0.1871 | 0.0614 | 0.2132 | 0.0722 | | R6 | 0.1813 | 0.0771 | 0.1565 | 0.0432 | |
| R7 | 0.6859 | 0.0813 | 0.6836 | 0.0642 | | R7 | 0.6852 | 0.0822 | 0.7038 | 0.0651 | |
| R8 | 0.3156 | 0.0644 | 0.2719 | 0.0755 | **↓, −13.9 | R8 | 0.4153 | 0.0574 | 0.4316 | 0.0581 | |
| R9 | 0.0088 | 0.0029 | 0.0080 | 0.0027 | | R9 | 0.0102 | 0.0025 | 0.0094 | 0.0020 | |
| F0 | 0.0362 | 0.0065 | 0.0341 | 0.0060 | | F0 | 0.0442 | 0.0074 | 0.0442 | 0.0047 | |
| F1 | 0.0396 | 0.0095 | 0.0358 | 0.0066 | | F1 | 0.0479 | 0.0118 | 0.0524 | 0.0108 | |
| ALL AGES B RIGHT-UPPER-OUTSIDE | | | | | | ALL AGES B RIGHT-UPPER-INSIDE | | | | | |
| R0 | 0.3190 | 0.0429 | 0.2884 | 0.0472 | *↓, −9.7 | R0 | 0.4309 | 0.0582 | 0.4226 | 0.0987 | |
| R1 | 0.0093 | 0.0020 | 0.0098 | 0.0024 | | R1 | 0.0098 | 0.0027 | 0.0105 | 0.0053 | |
| R2 | 0.9704 | 0.0073 | 0.9650 | 0.0109 | *↓, −0.5 | R2 | 0.9768 | 0.0075 | 0.9742 | 0.0140 | |
| R3 | 0.3282 | 0.0431 | 0.2978 | 0.0469 | *↓, −9.1 | R3 | 0.4408 | 0.0571 | 0.4330 | 0.0987 | |
| R4 | 0.0179 | 0.0045 | 0.0178 | 0.0050 | | R4 | 0.0162 | 0.0056 | 0.0196 | 0.0091 | |
| R5 | 0.7768 | 0.0739 | 0.8394 | 0.0654 | **↑, 8.0% | R5 | 0.7362 | 0.0983 | 0.8118 | 0.1002 | **↑, 10.3% |
| R6 | 0.1937 | 0.0455 | 0.2237 | 0.0836 | *↑, 15.5% | R6 | 0.1673 | 0.0546 | 0.1846 | 0.0682 | |
| R7 | 0.6510 | 0.0607 | 0.6875 | 0.0541 | **↑, 5.7% | R7 | 0.6326 | 0.0915 | 0.6849 | 0.0721 | **↑, 8.2% |
| R8 | 0.3097 | 0.0424 | 0.2787 | 0.0474 | *↓, −10.0 | R8 | 0.4211 | 0.0584 | 0.4121 | 0.0988 | |
| R9 | 0.0092 | 0.0020 | 0.0094 | 0.0023 | | R9 | 0.0099 | 0.0030 | 0.0104 | 0.0019 | |
| F0 | 0.0369 | 0.0077 | 0.0368 | 0.0052 | | F0 | 0.0436 | 0.0048 | 0.0462 | 0.0029 | |
| F1 | 0.0393 | 0.0067 | 0.0358 | 0.0063 | | F1 | 0.0535 | 0.0107 | 0.0499 | 0.0123 | |
| ALL AGES A RIGHT-FRONT-OUTSIDE | | | | | | ALL AGES A RIGHT-FRONT-INSIDE | | | | | |
| R0 | 0.2494 | 0.0571 | 0.2908 | 0.0586 | **↑, 16.9 | R0 | 0.3914 | 0.0479 | 0.3585 | 0.0507 | *↓, 8.2% |
| R1 | 0.0100 | 0.0032 | 0.0101 | 0.0036 | | R1 | 0.0156 | 0.0100 | 0.0113 | 0.0038 | |
| R2 | 0.9578 | 0.0153 | 0.9648 | 0.0117 | | R2 | 0.9588 | 0.0286 | 0.9677 | 0.0124 | |
| R3 | 0.2576 | 0.0589 | 0.2995 | 0.0592 | **↑, 16.3 | R3 | 0.4046 | 0.0473 | 0.3707 | 0.0515 | *↓, −8.4 |
| R4 | 0.0174 | 0.0064 | 0.0190 | 0.0056 | | R4 | 0.0241 | 0.0140 | 0.0215 | 0.0072 | |
| R5 | 0.7245 | 0.1231 | 0.7641 | 0.1001 | **↑, 5.4% | R5 | 0.7239 | 0.0874 | 0.7599 | 0.0844 | *↑, 5.0% |
| R6 | 0.2500 | 0.0481 | 0.2195 | 0.0718 | | R6 | 0.2547 | 0.0802 | 0.2435 | 0.0498 | |
| R7 | 0.5810 | 0.1051 | 0.6285 | 0.0898 | **↑, 8.3% | R7 | 0.5824 | 0.0967 | 0.6133 | 0.0827 | **↑, 5.33 |
| R8 | 0.2394 | 0.0575 | 0.2807 | 0.0574 | **↑, 17.6 | R8 | 0.3759 | 0.0524 | 0.3472 | 0.0510 | |
| R9 | 0.0082 | 0.0038 | 0.0086 | 0.0026 | | R9 | 0.0132 | 0.0048 | 0.0122 | 0.0023 | |
| F0 | 0.0352 | 0.0071 | 0.0378 | 0.0057 | | F0 | 0.0518 | 0.0074 | 0.0495 | 0.0055 | |
| F1 | 0.0395 | 0.0125 | 0.0413 | 0.0093 | | F1 | 0.0581 | 0.0103 | 0.0516 | 0.0099 | ***↓, −11.2 |
| ALL AGES B RIGHT-FRONT-OUTSIDE | | | | | | ALL AGES B RIGH-FRONT-INSIDE | | | | | |
| R0 | 0.2385 | 0.0789 | 0.2481 | 0.0776 | | R0 | 0.4102 | 0.0683 | 0.3892 | 0.0685 | |
| R1 | 0.0141 | 0.0076 | 0.0109 | 0.0055 | | R1 | 0.0120 | 0.0054 | 0.0126 | 0.0053 | |
| R2 | 0.9312 | 0.0548 | 0.9516 | 0.0252 | | R2 | 0.9696 | 0.0159 | 0.9662 | 0.0165 | |
| R3 | 0.2479 | 0.0786 | 0.2561 | 0.0777 | | R3 | 0.4196 | 0.0681 | 0.3992 | 0.0691 | |
| R4 | 0.0229 | 0.0126 | 0.0186 | 0.0092 | | R4 | 0.0213 | 0.0096 | 0.0242 | 0.0105 | |
| R5 | 0.6263 | 0.1118 | 0.7327 | 0.1022 | ***↑ | R5 | 0.6955 | 0.1220 | 0.7541 | 0.1015 | *↑, 8.3% |
| R6 | 0.2782 | 0.0915 | 0.2572 | 0.0728 | | R6 | 0.2185 | 0.0711 | 0.2166 | 0.0782 | |
| R7 | 0.4948 | 0.1065 | 0.5844 | 0.0845 | ***↑ | R7 | 0.5720 | 0.0994 | 0.6215 | 0.0876 | **↑, 8.7% |
| R8 | 0.2244 | 0.0824 | 0.2372 | 0.0793 | | R8 | 0.3981 | 0.0696 | 0.3766 | 0.0698 | |
| R9 | 0.0094 | 0.0026 | 0.0080 | 0.0025 | | R9 | 0.0094 | 0.0045 | 0.0100 | 0.0040 | |
| F0 | 0.0346 | 0.0059 | 0.0353 | 0.0071 | | F0 | 0.0487 | 0.0066 | 0.0476 | 0.0048 | |
| F1 | 0.0416 | 0.0096 | 0.0378 | 0.0097 | | F1 | 0.0607 | 0.0229 | 0.0552 | 0.0124 | |

5. Infiltration of ASESM into Human Skin and Reconstituted Human Three-Dimensional Culture Epithelium Model LabCyte (Trademark) EPI-MODEL Measured with Use of Raman Spectrometer

The infiltrations of the ASESM from the agent for external use into a human three-dimensional culture epithelium model (product name “LabCyte EPI-MODEL (registered trademark)”, Japan Tissue Engineering Co., Ltd.) and into human skin were examined.

As to the EPI-MODEL, a solution containing 10% (W/V) or 30% (W/V) ASESM in water (Milli-Q) or lotion (an aqueous solution containing 7% (V/V) butylene glycol, 1% (V/V) pentylene glycol, 4% (V/V) glycerin and 0.2% (V/V) phenoxyethanol) was added to the reconstituted human epithelium model (LabCyte EPI-MODEL) and this solution was aspirated off. After the EPI-MODEL was incubated at room temperature for 30 minutes, the measurement was performed with the use of an in vivo Raman spectrometer (n=3). As a control sample, the same sample was used except that it was treated with the same solution of 0% (W/V) ASESM (in water or lotion).

As to the human skin, the solution of 30% (W/V) ASESM in lotion (20 μl) was painted to the front arm of a 40-year-old woman. After the skin was air-dried for 60 minutes, the measurement was performed with the use of an in vivo Raman spectrometer (n=3). As a control sample, the same sample was used except that it was treated with the lotion of 0% (W/V) ASESM.

The infiltration profile of the Raman spectrum was measured from the skin surface towards the inside at intervals of 2 μm with the use of “River Diagnostics Skin Composition Analyzer Model 3510”. Twenty μl of the ASESM solution was applied to the area of 2 cm×2 cm square at the palm side of the front hand at various times, and then the change of infiltration profile caused by the ASESM lotion was measured 2 minutes and 10 minutes after the application.

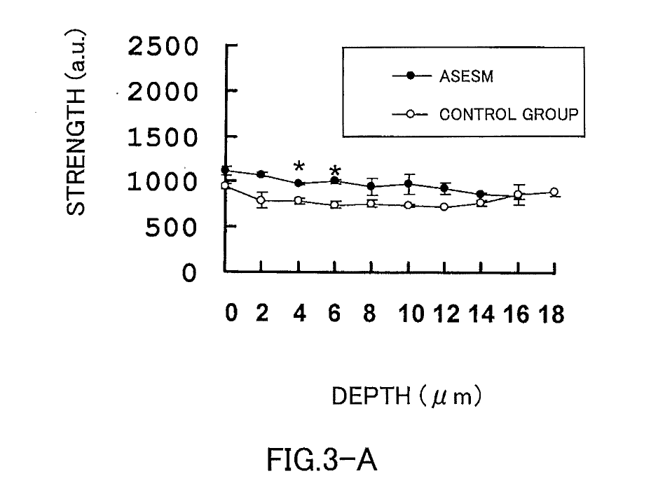

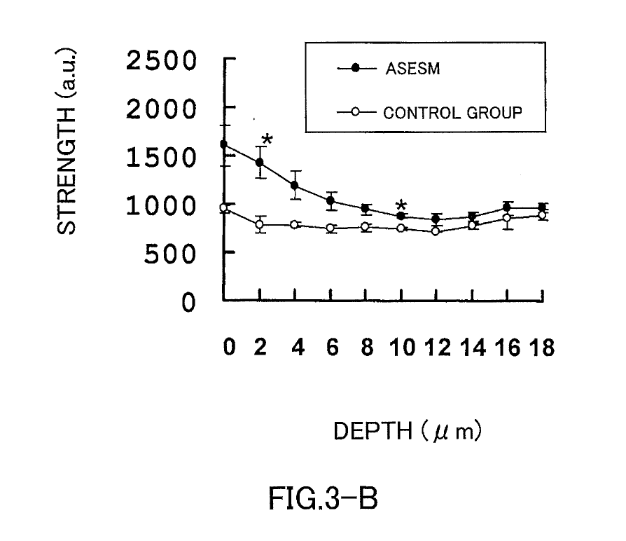

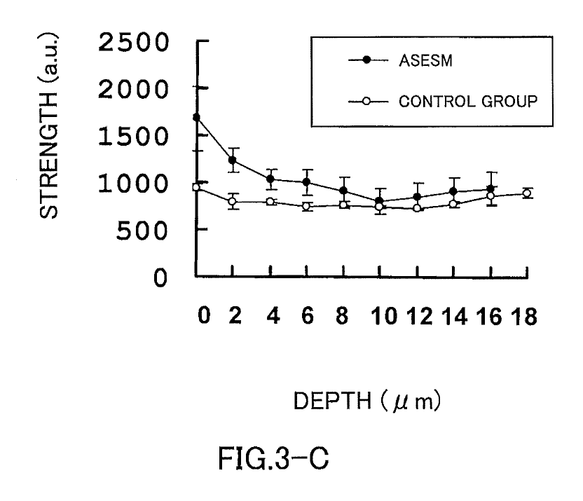

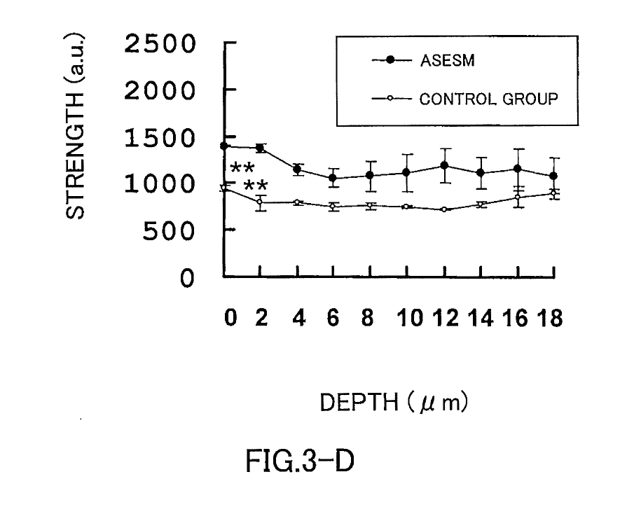

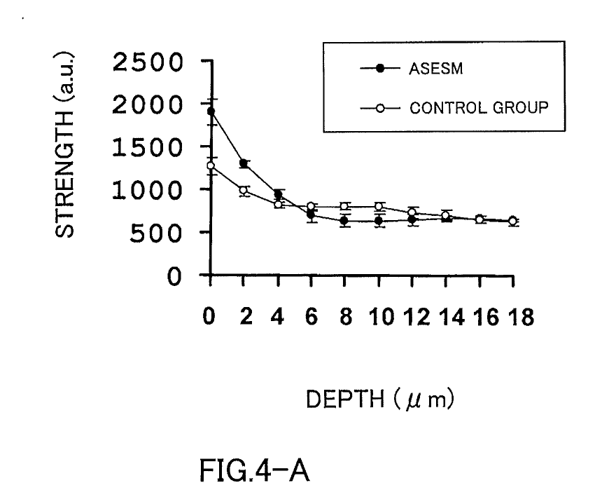

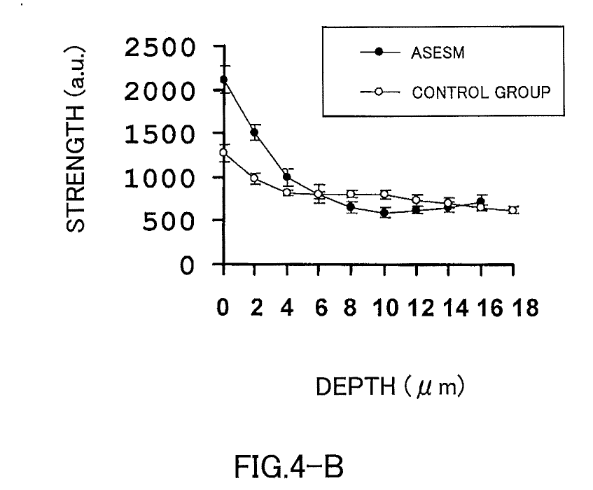

The result from the EPI-MODEL is shown in FIG. 3 and the result from the human skin is shown in FIG. 4. From these results, it was found that the ASESM was surely infiltrated into the skin.

In the present invention, by an in vivo effect of the ASESM, it was found that the combination of the ECM genes such as genes for type III collagen, decorin and MMP2, along with various sirtuin genes, particularly a mitochondrial sirtuin 3 (SIRT3) gene, were activated. The reduction of the water and the elasticity of the skin is one of the invisible signs of aging, but a conventional art regarding the improvement of both water and elasticity has not been found. In the present invention, it was indicated that affecting the cell of the skin by the egg shell membrane ingredient promoted the turnover of the protein in the cell and the blastogenesis of the cell, and the moderate activation of the sirtuin gene had a large potential in health of the skin. It was suggested that, by such actions, the water and elasticity of the skin were improved.

6. Production of Egg Shell Membrane-Containing Fine Powder

As an egg shell membrane-containing powder sample, a product name “EM Powder 300” from Kewpie Corporation pulverized by jet mill was used. As the jet mill, a single track jet mill (made by Seishin Enterprise Co., Ltd., FS-4) was used. The pulverization was performed under the air flow of 1.2 m³/min and the power of 11 kw so that the volume maximum particle diameter was through approximately 800 mesh (the opening was approximately 20 μm). According to the measurement of the particle diameter after the pulverization with the use of a laser diffraction-type particle size distribution measuring instrument (made by Seishin Enterprise Co., Ltd., LMS-30), the volume maximum particle diameter was 19.6 μm and the volume average particle diameter was 5.8 μm.

7. Effect of Intake of Egg Shell Membrane-Containing Powder on Sirtuin Gene Expression in Mouse

After a male C57BL6/J mouse at 6 to 7 weeks of age was fasted from the day before, 0.5 mg or 1 mg of a supplement for experiment only containing the egg shell membrane fine powder and egg shell calcium as an active ingredient (“8φCR 200 mg”, 37.50% egg shell membrane-containing fine powder (800 mesh) produced as described above (75.0 mg); 11.75% egg shell calcium (Kewpie Corporation) (23.5 mg); 43.75% lactose (Glanbia plc) (87.5 mg); 5.00% corn protein (Kobayashi Perfumery Co., Ltd.) (10.0 mg); 2.00% rape hardened oil (Kawaken Fine Chemicals Co., Ltd.) (4.0 mg)), or 1 mg of a control tablet only containing an excipient as a control (“9pCR 250 mg”, 93.00% lactose (232.5 mg); 5.00% corn protein (12.5 mg); 2.00% rape hardened oil (5.0 mg)) (all tablets are pulverized in a mortar) was suspended in 100 μL of a jelly for drug administration in animal (product name MediGel Sucralose, Japan SLC, Inc.), and then the entire thereof was directly administered to the stomach of the lightly etherized mouse with the use of a sonde (n=1 for each). After 16 hours, the mouse was dissected and the expression of Sirt 1 to 4 genes within the cells in the tissues of kidney, liver, soleus muscle, gastrocnemius muscle, hippocampus, brown fat (BAT) and white fat (WAT) was evaluated by quantitative real time PCR in the same way as described above.

The result is shown in Table 6. The tendency was found that the expression of sirt 3 gene in the kidney, sirt 1 genes in the liver, gastrocnemius muscle and hippocampus, sirt 1 and sirt 3 genes in the BAT, and all sirt 1 to 4 genes in WAT was increased.

TABLE 6

| | AMOUNT OF SUPPLEMENT | | | | | | | ASTROC- |
|---|---|---|---|---|---|---|---|---|
| | CONTAINING EGG SHELL | HIPPO- | | | BROWN | WHITE | SOLEUS | NEMIUS |
| | MEMBRANE/INDIVIDUAL | CAMPUS | LIVER | KIDNEY | FAT | FAT | MUSCLE | MUSCLE |
| Sirt. 1 | 0.5 mg | ↑1.57-FOLD | ↑1.24-FOLD | ↓0.91-FOLD | ↑1.21-FOLD | ↑2.01-FOLD | ↓0.94-FOLD | ↑1.18-FOLD |
| | 1.0 mg | ↑1.17-FOLD | ↑1.30-FOLD | — | ↑1.83-FOLD | ↑1.75-FOLD | — | ↑1.11-FOLD |
| Sirt. 2 | 0.5 mg | ↓0.93-FOLD | →0.98-FOLD | ↓0.94-FOLD | ↓0.94-FOLD | ↑1.29-FOLD | →0.97-FOLD | ↓0.87-FOLD |
| | 1.0 mg | ↓0.82-FOLD | →1.02-FOLD | — | ↑1.37-FOLD | ↑1.23-FOLD | — | ↓0.79-FOLD |
| Sirt. 3 | 0.5 mg | →0.98-FOLD | ↓0.80-FOLD | ↑1.21-FOLD | ↑1.11-FOLD | ↑1.21-FOLD | ↓0.79-FOLD | ↓0.86-FOLD |
| | 1.0 mg | ↑1.03-FOLD | ↓0.95-FOLD | | ↑1.18-FOLD | ↑1.39-FOLD | — | ↓0.68-FOLD |
| Sirt. 4 | 0.5 mg | ↓0.88-FOLD | ↓0.84-FOLD | ↓0.95-FOLD | ↓0.81-FOLD | | ↓0.95-FOLD | ↑1.50-FOLD |
| | 1.0 mg | ↓0.71-FOLD | ↓0.94-FOLD | | | | ↓0.87-FOLD | ↑1.79-FOLD |

8. Disposition of Egg Shell Membrane Ingredient

A nitrogen-containing compound such as a protein is blended with lithium carbonate and is irradiated with neutrons to be labeled with tritium produced through a Li⁶ (n, α)³H reaction. Through the use of this, the disposition of the egg shell membrane-containing powder labeled with tritium when being orally administered to a mouse was examined as follows.

<Labeling of Egg Shell Membrane>

After 0.32 g of egg shell membrane-containing powder (“EM powder”, Kewpie Corporation) and 0.65 g of lithium carbonate were sufficiently blended with each other and encapsulated under reduced pressure in a silica tube, the blended material was irradiated with neutrons for 20 minutes in Nuclear Science Research Institute of Japan Atomic Energy Agency (JRR4 nuclear reactor). The irradiated sample was removed from the silica tube, and water was mixed therewith to dissolve the unreacted lithium carbonate. The egg shell membrane powder insoluble in water was filtered and recovered. The egg shell membrane powder was washed with water until the radioactivity of the filtrate was sufficiently decreased, so that the tritium not combined to the egg shell membrane was removed.

<Experimental Animal>

After C57BL/6J mice at 6 weeks of age purchased from Oriental Yeast Co., Ltd. were preliminarily fed for approximately 1 week (under the environmental condition of temperature 23±2° C., relative humidity 55±10%, and 12 hour light/dark cycle), an experiment was performed at 7 weeks of age. The mice were housed in each metabolism cage (Metabolica MM) (86.5 cm²×14.5 cm, approximately 2,000 cm³ space) from Sugiyama-Gen Co., Ltd. and were given free access to a solid feed (MF, Oriental Yeast Co., Ltd.) and tap water.

<Administration Method>

The labeled egg shell membrane-containing powder suspended in water was orally administered by force in a single dose to the stomach of the mouse fasted for 16 hours before the administration with the use of a plastic disposable sonde. The radioactivity after the administration was adjusted to approximately 4.5 MBq/kg (122 mCi/kg) body weight and the dose was adjusted to 250 mg/kg body weight.

<Radioactivity Measurement>

A radioactivity measurement was performed with the use of a liquid scintillation counter (Packard, 2200CA) in which a scintillator was added to a prepared sample for radioactivity measurement. The correction of the quenching was done based on an external standard ratio method.

<Measurement of Radioactive Concentration in Blood>

Five ml of blood was collected from the tail vein 0.25, 0.5, 1, 2, 4, 6, 9, 12 and 24 hours after and 2, 3, 4, 5 and 6 days after the administration of the labeled egg shell membrane-containing powder. To this sample, 1 ml of a tissue solubilizer (Soluene-350 (Perkin Elmer)/isopropyl alcohol (1:1)) was added, and the mixture was heated and shaken at 50° C. for 3 hours. Subsequently, to the heated and shaken mixture, 500 ml of 30% aqueous hydrogen peroxide was added. To this sample, 10 ml of a scintillator (Hionic fluor, PerkinElmer) was added, and the radioactivity was measured.

<Excretion of Radiogen in Urine and Feces>

After the administration of the labeled compound, from the mouse housed in the metabolism cage (Metabolica MM, Sugiyama-Gen Co., Ltd.), the urine and feces were separately collected every single day for 6 days after the administration. To a precisely weighed part of the collected feces, 2 ml of a tissue solubilizer was added, and the mixture was heated at 50° C. for 3 to 4 hours. Subsequently, to the heated mixture, 1 ml of isopropanol was added, and the mixture was heated at 50° C. for 2 hours. To this sample, 0.5 ml of 30% aqueous hydrogen peroxide was added and 10 ml of a scintillator (Hionic fluor, PerkinElmer) was added, and the radioactivity was measured. To 1 ml of the urine in each fraction, 5 ml of a scintillator (Ultima Gold LLT) was added, and the radioactivity was measured.

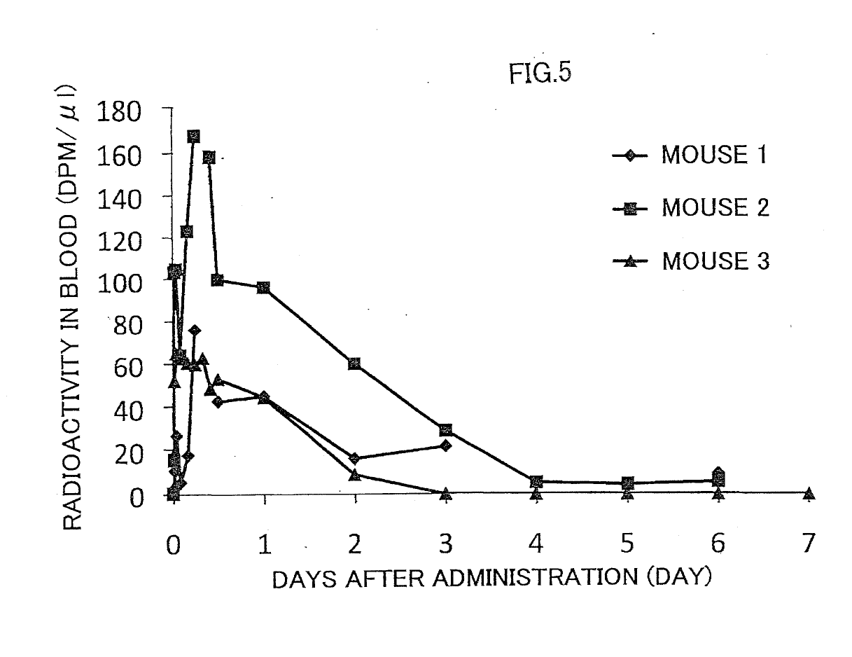

The result of the radioactive concentration in blood after the oral administration of the egg shell membrane to the mouse is shown in FIG. 5. The radioactive concentration in blood in accordance with the time course after the administration of the tritium labeled egg shell membrane was shown. The radioactive concentration in blood reached a maximum within 24 hours after the administration but subsequently decreased to the original radioactivity level within 3 to 4 days.

The result of the radioactive concentrations in the urine and feces is shown in Table 7. The radioactivity in the urine and feces up to 3 days after the administration was 61.05% of the whole dose.

TABLE 7

| | ³H RADIOACTIVITY (RATIO TO DOSE (%)) | | |
|---|---|---|---|
| | DAY 1 | DAY 2 | DAY 3 |
| IN FECES | 14.78 | 33.02 | 11.93 |
| IN URINE | 0.47 | 0.50 | 0.35 |

<Migration of Radiogen to Tissue of Whole Body>

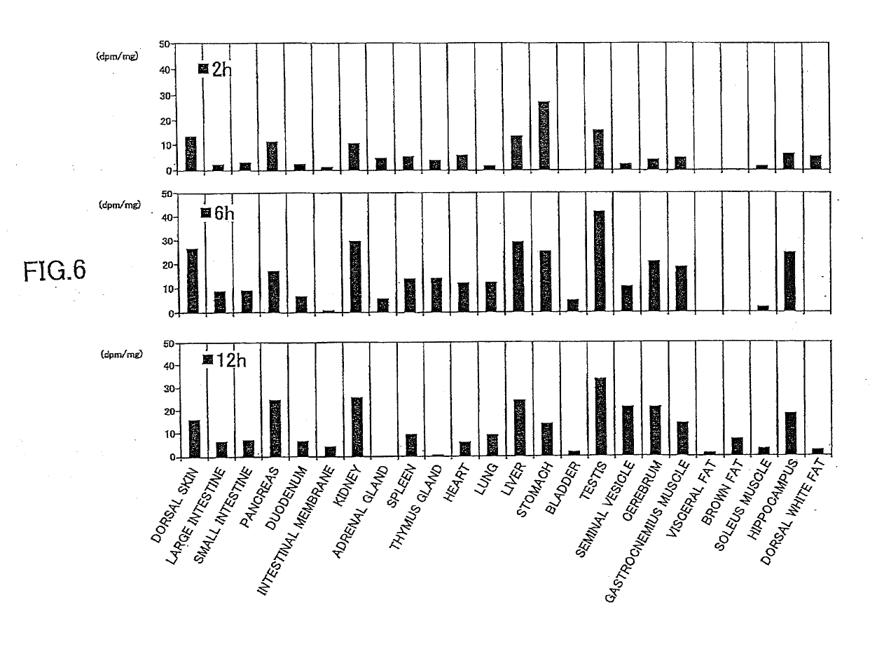

In the same way as described above, the 5,568,000 dpm tritium labeled egg shell membrane was administered to each stomach of 3 mice with the use of a sonde. Two hours, 6 hours and 12 hours after the administration, a part or whole of the tissue was enucleated from each individual, and weighed. To each tissue, 2 ml of a tissue solubilizer (Soluene-350) was added, and the mixture was incubated at 60° C. for 3 hours. To this sample, 0.5 ml of 30% aqueous hydrogen peroxide was added and 10 ml of a scintillator (Hionic fluor) was added. After the mixture was incubated at room temperature for 1 hour, the radioactivity was measured with the use of a liquid scintillation counter. The result is shown in Table 8 and FIG. 6.

TABLE 8

| | 2 h | 6 h | 12 h |
|---|---|---|---|
| DORSAL SKIN | 14.39827957 | 26.5335 | 16.06 |
| LARGE INTESTINE | 3.423908524 | 8.58844221 | 6.5 |
| SMALL INTESTINE | 4.218064516 | 8.83794393 | 7.12 |
| PANCREAS | 12.13833922 | 16.8112288 | 24.69 |
| DUODENUM | 3.623255814 | 6.47457627 | 6.57 |
| INTESTINAL | 2.345011601 | 0.75178571 | 4.33 |
| MEMBRANE | | | |
| KIDNEY | 11.43458498 | 29.500316 | 25.59 |
| ADRENAL GLAND | 5.806666667 | 5.34285714 | 0 |
| SPLEEN | 6.379558011 | 13.558216 | 9.46 |
| THYMUS GLAND | 5.085465116 | 13.5094737 | 0.18 |
| HEART | 6.943851508 | 11.6870663 | 5.93 |
| LUNG | 2.68342246 | 11.8900826 | 8.89 |
| LIVER | 14.24795918 | 28.8658174 | 24.23 |
| STOMACH | 27.68076923 | 24.9255708 | 13.85 |
| BLADDER | 0 | 4.62395833 | 1.5 |
| TESTIS | 16.60234375 | 41.5360656 | 33.31 |
| SEMINAL VESICLE | 3.254893617 | 10.1778351 | 21.19 |
| CEREBRUM | 5.092879257 | 20.5594406 | 21.05 |
| GASTROCNEMIUS | 5.785743381 | 18.1498305 | 13.87 |
| MUSCLE | | | |
| VISCERAL FAT | 0 | 0 | 0.8 |
| BROWN FAT | 0 | 0 | 6.79 |
| SOLEUS MUSCLE | 2.421428571 | 1.84 | 2.73 |
| HIPPOCAMPUS | 7.248201439 | 24.0738462 | 17.88 |
| DORSAL WHITE FAT | 6.22173913 | 0.03995215 | 2.21 |
| (dpm/mg) | | | |

It became clear that the egg shell membrane ingredient was highly distributed in various tissues of the whole body, particularly in skin, kidney, liver, testis (ovarian in case of female) and brain (for example, hippocampus). These tissues closely correspond with the parts in which SIRT1 and SIRT3 are highly expressed.

9. Production of Pharmaceutical Composition (Tablet)

(1) Production of Granule for Compression

A raw material mixture was prepared through the blending of the egg shell membrane-containing fine powder (800 mesh) produced as described above: 20.0 parts by mass, “Waxy a” made by Nisshoku Co., Ltd.: 10.0 parts by mass, “Pine Fiber” made by Matsutani Chemical Industry Co., Ltd.: 20.0 parts by mass, lactose (Meggle Japan Co., Ltd.): 25.9 parts by mass, egg shell calcium (“Calhope” made by Kewpie Corporation): 10 parts by mass, β-carotene: 5.0 parts by mass, vitamin B: 20.05 parts by mass, vitamin E: 0.05 parts by mass and niacin: 2.0 parts by mass, with the use of a V-type blender. Subsequently, with the raw material mixture (93.0 parts by mass), ethyl alcohol (15 parts by mass) was blended. The obtained mixture was granulated with the use of a wet granulator and then dried at temperature 50° C. for approximately 16 hours, with the result that a granule for compression was produced.

(2) Compression

Next, with the granule for compression (100 parts by mass), vitamin C (9 parts by mass) and sucrose fatty acid ester (1 parts by mass) were blended in such a ratio, and from the obtained mixture, 200 mg of one uncoated tablet was produced with the use of a compressor.

(3) Protective Coating

Next, to the surface of the uncoated tablet, an aqueous solution of “Shellac” made by Gifu Shellac Manufacturing Co., Ltd. was painted with the use of a coating device, and the resultant was dried at temperature 40° C. for 2 hours, with the result that a protective coating applied tablet (protectively coated tablet) was obtained.

(4) Sugar Coating

The sufficiently dried surface of the protectively coated tablet was coated with a paste A for sugar coating (a paste in which 70 parts by mass granulated sugar, 3 parts by mass arabic gum, 4 parts by mass gelatin, 3 parts by mass egg shell calcium and 65 parts by mass water were blended) with the use of a sugar coating device, and then the resultant was dried at temperature approximately 40° C. for approximately 4 hours. Subsequently, a paste B for sugar coating was prepared which was from the paste A for sugar coating diluted with the addition of water. Then, the tablet surface that had undergone the coating process using the paste A for sugar coating and the drying process was coated with the paste B for sugar coating with the use of a sugar coating device, and then the resultant was dried at temperature approximately 40° C. for approximately 4 hours. In this way, a tablet coated with a paste for sugar coating (sugar coated tablet) was obtained.

(5) Coloring

To the surface of the sugar coated tablet, a coloring liquid containing “SR Red K3” made by San-Ei Gen F.F.I., Inc. was painted, and then the resultant was dried at temperature 40 to 50° C. for 4 hours, with the result that a red-colored tablet (colored tablet) was produced.

(6) Glazing

A glazing was performed in which, to the surface of the colored tablet, carnauba wax was applied. The mass of one tablet obtained in this way was 400 mg, in which approximately 40 mg of the egg shell membrane ingredient was contained.

(7) Screening-Measuring-Packaging

The glazed tablet was subjected to a screening for removing the defective, measured after the product inspection, and packaged in a duplex wall bag with a desiccating agent enclosed. Note that the tablet had a sufficient hardness and shape retentivity to avoid the deformation or disintegration during screening, inspection and packaging of the tablet, and had an excellent suitability to handling.

What is claimed is:

1. A method of activating gene expression of a sirtuin gene in a subject in need thereof comprising the step of administering to the subject an effective amount of a composition comprising a powder containing eggshell membrane or a soluble component of eggshell membrane.

2. The method according to claim 1, wherein the method comprises administering the composition topically, and wherein the composition further comprises a diluting agent.

3. The method according to claim 2, wherein the composition comprises a soluble component of eggshell membrane.

4. The method according to claim 3, wherein the soluble component of eggshell membrane is a hydrolysate of the eggshell membrane.

5. The method according to claim 4, wherein the composition further comprises one or more active ingredients selected from the group consisting of antiphlogistic agents, anti-inflammatory drugs, melanin production suppressants, melanin reducing agents, hair bleaching agents, melanin excretion promoters, cell activators, antioxidants, anti-oxidizing agents, keratolytic-release agents, sebum suppressors, moisturizing agents, emollient agents, sebum secretion suppressors/promoters, ultraviolet absorbing agents, antiperspirants, blood circulation improving agents, exfoliating cleansers/softening agents, skin whitening agents, anti-allergic drugs, steroid hormones, immunosuppressant, and antibiotics.

6. The method according to claim 1, wherein the method comprises administering the composition orally, and wherein the composition further comprises a diluting agent.

7. The method according to claim 6, wherein the composition comprises a powder containing eggshell membrane.

8. The method according to claim 7, wherein the powder containing eggshell membrane has a mean volume particle diameter of about 5 μm to about 40 μm.

9. The method according to claim 6, wherein the composition is in the form of a tablet and comprises one or more of bonding agents, disintegrating agents, lubricants, and nutritive components.

10. The method according to claim 9, wherein the diluting agent is modified starch or lactose.

11. The method according to claim 7, wherein the diluting agent is present in an amount of 0.5 to 3.0 mass times the powder containing eggshell membrane.

12. A method of enhancing the expression of one or more genes selected from the group consisting of sirtuin 1, sirtuin 2, sirtuin 3, sirtuin 4, sirtuin 5, sirtuin 6, and sirtuin 7 in a subject in need thereof, comprising the step of administering to the subject an effective amount of a composition comprising a powder containing eggshell membrane, or a soluble component of eggshell membrane.


SEQUENCE LISTING (47 sequences)

SEQ ID NO: 1 - DNA, 24 bp, Artificial Sequence
  Mm_Sirt1-F-2
tcctgacttcagatcaagagacgg

SEQ ID NO: 2 - DNA, 20 bp, Artificial Sequence
  Mm_Sirt1-R-2
gtgacacagagacggctgga

SEQ ID NO: 3 - DNA, 19 bp, Artificial Sequence
  Mm_Sirt2-F
cgggcagttcaagccaacc

SEQ ID NO: 4 - DNA, 21 bp, Artificial Sequence
  Mm_Sirt2-R
ccagcgtgtctatgttctgcg

SEQ ID NO: 5 - DNA, 21 bp, Artificial Sequence
  Mm_Sirt3-F
gacgggcttgagagagcatct

SEQ ID NO: 6 - DNA, 19 bp, Artificial Sequence
  Mm_Sirt3-R
gtccgccatcacatcagcc

SEQ ID NO: 7 - DNA, 20 bp, Artificial Sequence
  Mm_Sirt4-F
ccaacccaacccagcacact

SEQ ID NO: 8 - DNA, 22 bp, Artificial Sequence
  Mm_Sirt4-R
cacagttcaggcacaggactct

SEQ ID NO: 9 - DNA, 22 bp, Artificial Sequence
  Mm_Sirt5-F
tgtgacctgtgtctagtggtgg

SEQ ID NO: 10 - DNA, 23 bp, Artificial Sequence
  Mm_Sirt5-R
gcttcaggaagagttttcccaca

SEQ ID NO: 11 - DNA, 20 bp, Artificial Sequence
  Mm_Sirt6-F
gacttcgggcctgtagaggg

SEQ ID NO: 12 - DNA, 20 bp, Artificial Sequence
  Mm_Sirt6-R
gacagacaggtctgcggtcc

SEQ ID NO: 13 - DNA, 19 bp, Artificial Sequence
  Mm_Sirt7-F
gcagtggaccccgaaggat

SEQ ID NO: 14 - DNA, 21 bp, Artificial Sequence
  Mm_Sirt7-R
gaagattggatcctgccaccg

SEQ ID NO: 15 - DNA, 20 bp, Artificial Sequence
  mGAPDH-Left
agcttgtcatcaacgggaag

SEQ ID NO: 16 - DNA, 21 bp, Artificial Sequence
  mGAPDH-Right
tttgatgttagtggggtctcg

SEQ ID NO: 17 - DNA, 21 bp, Artificial Sequence
  Mouse Col 1a1 left
catgttcagctttgtggacct

SEQ ID NO: 18 - DNA, 20 bp, Artificial Sequence
  Mouse Col 1a1 right
gcagctgacttcagggatgt

SEQ ID NO: 19 - DNA, 20 bp, Artificial Sequence
  Mouse Col 3a1 left
tcccctggaatctgtgaatc

SEQ ID NO: 20 - DNA, 20 bp, Artificial Sequence
  Mouse Col 3a1 right
tgagtcgaattggggagaat

SEQ ID NO: 21 - DNA, 21 bp, Artificial Sequence
  Mouse Col 4a1 left
ttaaaggactccagggaccac

SEQ ID NO: 22 - DNA, 19 bp, Artificial Sequence
  Mouse Col 4a1 right
cccactgagcctgtcacac

SEQ ID NO: 23 - DNA, 20 bp, Artificial Sequence
  Mouse MMP-2-2 left
ggagaaggctgtgttcttcg

SEQ ID NO: 24 - DNA, 18 bp, Artificial Sequence
  Mouse MMP-2-2 right
aggctggtcagtggcttg

SEQ ID NO: 25 - DNA, 21 bp, Artificial Sequence
  Mouse MMP-3 left
ttgttctttgatgcagtcagc

SEQ ID NO: 26 - DNA, 18 bp, Artificial Sequence
  Mouse Hsp47 left
gcggactgcctggtaaac

SEQ ID NO: 27 - DNA, 18 bp, Artificial Sequence
  Mouse Hsp47 left
gaaggagagagcgcatgg

SEQ ID NO: 28 - DNA, 18 bp, Artificial Sequence
  Mm_Eln-F
ctcctccatcccgcgcag

SEQ ID NO: 29 - DNA, 19 bp, Artificial Sequence
  Mm_Eln-R
tcccagaagtccggcacct

SEQ ID NO: 30 - DNA, 19 bp, Artificial Sequence
  mouse decorin left
gaggagaagtgaggggaga

SEQ ID NO: 31 - DNA, 27 bp, Artificial Sequence
  mouse decorin right
catgattatctcatgtattttcacgac

SEQ ID NO: 32 - DNA, 19 bp, Artificial Sequence
  mouse Has2 left
ggcggaggacgagtctatg

SEQ ID NO: 33 - DNA, 24 bp, Artificial Sequence
  mouse Has2 right
acacatagaaacctctcacaatgc

SEQ ID NO: 34 - DNA, 24 bp, Artificial Sequence
  Mm_Tgfb1-F
ggataccaactattgcttcagctc

SEQ ID NO: 35 - DNA, 23 bp, Artificial Sequence
  Mm_Tgfb1-R
gtgtccaggctccaaatataggg

SEQ ID NO: 36 - DNA, 21 bp, Artificial Sequence
  Mm_Tgfb3-F
cggatgagcacatagccaagc

SEQ ID NO: 37 - DNA, 22 bp, Artificial Sequence
  Mm_Tgfb3-R
ccagacccaagttggactctct

SEQ ID NO: 38 - DNA, 20 bp, Artificial Sequence
  mouse SMPD1 left
tgctgagaatcgaggagaca

SEQ ID NO: 39 - DNA, 19 bp, Artificial Sequence
  mouse SMPD1 right
gaccggccagagtgttttc

SEQ ID NO: 40 - DNA, 18 bp, Artificial Sequence
  mouse AQP-3 left
ctggggaccctcatcctt

SEQ ID NO: 41 - DNA, 18 bp, Artificial Sequence
  mouse AQP-3 right
tggtgaggaagccaccat

SEQ ID NO: 42 - DNA, 21 bp, Artificial Sequence
  mouse MMP-3 left
ttgttctttgatgcagtcagc

SEQ ID NO: 43 - DNA, 18 bp, Artificial Sequence
  Mouse MMP-3 right
gatttgcgccaaaagtgc

SEQ ID NO: 44 - DNA, 24 bp, Artificial Sequence
  Mouse Has1 left
aaagagaacaagacggagaagaga

SEQ ID NO: 45 - DNA, 18 bp, Artificial Sequence
  Mouse Has1 right
ctgagggctttggcatgt

SEQ ID NO: 46 - DNA, 18 bp, Artificial Sequence
  mouse Sptlc1 left
ggtgctggtggagatggt

SEQ ID NO: 47 - DNA, 23 bp, Artificial Sequence
  mouse Sptlc1 right
ggattccttccaaaataagatgg